(19) United States
(12) Patent Application Publication
(10) Pub. No.: US 2012/0308642 A1
(43) Pub. Date: Dec. 6, 2012

(54) INHIBITING HEPATITIS C VIRAL REPLICATION WITH SIRNA COMBINATIONS

(75) Inventors: Srikanta DASH, New Orleans, LA (US); Tarun K. MANDAL, Kenner, LA (US); Partha K. CHANDRA, New Orleans, LA (US); Anup KUNDU, Kenner, LA (US); Sidhartha HAZARI, Overland Park, KS (US)

(73) Assignees: XAVIER UNIVERSITY OF LOUISIANA, New Orleans, LA (US); THE ADMINISTRATORS OF THE TULANE EDUCATIONAL FUND, New Orleans, LA (US)

(21) Appl. No.: 13/483,025

(22) Filed: May 29, 2012

Related U.S. Application Data
(60) Provisional application No. 61/519,725, filed on May 27, 2011.

(51) Int. Cl. A61K 31/713; C07H 21/02; A61P 31/14; A61K 9/127; B82Y 5/00

(52) U.S. Cl. 424/450; 514/44 A; 536/24.5; 977/800; 977/907

(57) ABSTRACT

Compositions are disclosed of combinations of small interfering RNAs (siRNA) that can inhibit the replication of hepatitis C virus (HCV) in liver cells, along with methods of co-administering the siRNAs to subjects. Further, methods are disclosed for improving the delivery of nucleic acids to the liver.

### CROSS-REFERENCE TO RELATED APPLICATIONS

This application claims priority to U.S. Provisional Patent Application No. 61/519,725, filed May 27, 2011, the contents of which are hereby incorporated herein.

### STATEMENT OF FEDERAL FUNDING

This invention was made with government support under Grant 1R01CA127481 awarded by the National Cancer Institute. The government has certain rights in the invention.

### BACKGROUND OF THE INVENTION

Hepatitis C, caused by the hepatitis C virus (“HCV”), is a blood borne infectious disease that affects the liver. The majority of people infected with this virus end up with a chronic infection. Due to the asymptomatic nature of HCV infection, many individuals do not even realize that they are infected until the virus significantly damages the liver decades later. The problem is considered so serious that in May 2012, the Centers for Disease Control and Prevention (“CDC”) recommended that all U.S. residents born between 1945 and 1965 be tested for the disease.

Chronic HCV infection causes liver cirrhosis and cancer. At present there is no treatment for liver cirrhosis or cancer, so most of these patients seek a liver transplantation. Currently, there is no effective vaccine available for HCV infection. The only FDA approved treatment is alpha-interferon (IFN-α) in combination with ribavirin, but the majority of these patients develop resistance to this combination therapy. There are approximately 170 million people infected with HCV worldwide, and the CDC estimates that 3.2 million people in the United States have chronic infections. The number of new HCV infections each year is increasing at a very high rate in developing nations due to blood transfusions from unscreened donors. HCV infection represents a significant worldwide public health problem, imposing high social, medical and economic burdens. The development of alternative therapeutic strategies to prevent or to reduce hepatitis C virus infection and consequent disease is therefore a significant public health priority.

IFNα antiviral mechanisms target the 5′ UTR region of the HCV genome, which is utilized by the virus to translate protein by an internal ribosome entry site (“IRES”) mechanism in the infected cells. Although the sequence is highly conserved among different HCV genotypes, half of chronic hepatitis C patients do not respond to interferon. Thus, interferon therapy does not provide an adequate means to address the problem of HCV infection.

One proposed alternative therapeutic method is the use of RNA interference, and specifically small interfering RNAs (“siRNAs”). Progress in the use of siRNA-based antiviral strategies for HCV has been slow due to the number of challenges related to in vivo delivery of siRNA molecules to liver hepatocytes to achieve long lasting clearance of virus.

One approach to delivering therapeutic siRNAs to the liver is the use of non-viral vectors. Non-viral vectors are less toxic and less immunogenic. They have the potential to be administered repeatedly and are easy to produce in large quantities. Since siRNAs in cells for only a few days after intracellular delivery, however, repeated treatment of siRNA formulations may be required to maintain high intracellular levels sufficient to clear the viral infection. Development of escape mutations in the viral genome has been reported in the siRNA based antiviral approach, particularly when single siRNA targets were used. It would be desirable to have siRNAs that better reduce HCV replication in infected individuals. Further, it would be desirable to have better methods for delivering siRNAs or other agents to the liver.

### PARTIES TO JOINT RESEARCH AGREEMENT

Not applicable.

### REFERENCE TO SEQUENCE LISTING OR TABLE SUBMITTED ON COMPACT DISC AND INCORPORATION-BY-REFERENCE OF THE MATERIAL [SPECIFY NUMBER OF DISCS AND FILES ON EACH]

Not applicable.

### BRIEF SUMMARY OF THE INVENTION

In a first set of embodiments, the invention provides compositions comprising at least a first and a second isolated small interfering RNA (siRNA), each siRNA comprising a sense sequence having a 5′ end and a 3′ end, and a complementary antisense sequence having a 5′ and a 3′ end, wherein the sense sequence of the first isolated siRNA comprises 19-24 contiguous nucleotides of a sequence selected from the group consisting of:

(a)
(SEQ ID NO: 29)
5′-GCCUUGUGGUACUGCCUGAUAGGG-3′;
(b)
(SEQ ID NO: 30)
5′-CCCCGGGAGGUCUCGUAGACCGUG-3′;
(c)
(SEQ ID NO: 31)
5′-GAGGUCUCGUAGACCGUGCACCAU-3′;
(d)
(SEQ ID NO: 32)
5′-ACCGUGCACCAUGAGCACAAAUCC-3′;
(e)
(SEQ ID NO: 33)
5′-AACCUCAAAGAAAAACCAAAAGAA-3′;

and, wherein the sense strand of the second isolated siRNA is selected from the group consisting of sequences (a)-(e), provided that the sense sequence of the first isolated siRNA and the sense sequence of the second isolated siRNA are not the same. In some embodiments, the sense sequence and said antisense sequence of said first siRNA and of said second siRNA, respectively, have a two T overhang on said 3′ ends. In some embodiments, the sense sequence, with overhang, of the first siRNA consists of a sequence selected from the group consisting of SEQ ID NO:11, SEQ ID NO:15, SEQ ID NO:17, SEQ ID NO:19, and SEQ ID NO:23, provided that if the sense sequence selected is SEQ ID NO:17, the sense sequence selected for the sense sequence of the second siRNA is not SEQ ID NO:19. In some embodiments, the sense sequence, with overhang, of the second siRNA consists of a sequence selected from the group consisting of SEQ ID NO:11, SEQ ID NO:15, SEQ ID NO:17, SEQ ID NO:19, and SEQ ID NO:23. In some embodiments, the sense and antisense sequences of the first siRNA have the sequences SEQ ID NO:17 and SEQ ID NO:18, respectively, and the sense and antisense sequences of the antisense sequences of the second siRNA have the sequences of SEQ ID NO:23 and SEQ ID NO:24, respectively. In some embodiments, the first siRNA and the second siRNA are condensed with protamine sulfate. In some embodiments, the first siRNA and the second siRNA are complexed to or encapsulated in a nanosome. In some embodiments, the nanosome comprises a cationic lipid. In some embodiments, the cationic lipid is selected from the group consisting of 3β-[N-(N′,N′-dimethylaminoethane)-carbamoyl]cholesterol hydrochloride (DC-cholesterol); 1,2-dioleoyl-3-trimethylammonium-propane (DOTAP); 1,2-dioleoyl-3-dimethylammonium-propane (DODAP); dimethyldioctadecylammonium (DDAB); 1,2-dilauroyl-sn-glycero-3-ethylphosphocholine (Ethyl-PC), or 1,2-di-O-octadecenyl-3-trimethylammonium propane (DOTMA). In some embodiments, the cationic lipid is DOTAP. In some embodiments, the composition further comprises a neutral lipid. In some embodiments, the neutral lipid is cholesterol. In some embodiments, the composition further comprises a sugar. In some embodiments, the sugar is selected from the group consisting of sucrose, trehalose, lactose and mannitol. In some embodiments, the sugar is trehalose. In some embodiments, the composition is a powder. In some embodiments, the composition is a lyophilized powder. In some embodiments, the composition is a spray dried powder.

In a further set of embodiments, the invention provides methods of inhibiting replication of hepatitis C virus (HCV) in a subject in need thereof, said method comprising co-administering to the subject a first and a second isolated small interfering RNA (siRNA), each siRNA comprising a sense sequence having a 5′ end and a 3′ end, and a complementary antisense sequence having a 5′ and a 3′ end, wherein the sense sequence of the first isolated siRNA comprises 19-24 contiguous nucleotides of a sequence selected from the group consisting of:

(a)
(SEQ ID NO: 29)
5′-GCCUUGUGGUACUGCCUGAUAGGG-3′;
(b)
(SEQ ID NO: 30)
5′-CCCCGGGAGGUCUCGUAGACCGUG-3′;
(c)
(SEQ ID NO: 31)
5′-GAGGUCUCGUAGACCGUGCACCAU-3′;
(d)
(SEQ ID NO: 32)
5′-ACCGUGCACCAUGAGCACAAAUCC-3′;
(e)
(SEQ ID NO: 33)
5′-AACCUCAAAGAAAAACCAAAAGAA-3′;

and, wherein the sense strand of the second isolated siRNA is selected from the group consisting of sequences (a)-(e), provided that the sense sequence of said first isolated siRNA and the sense sequence of said second isolated siRNA are not the same, thereby inhibiting the replication of HCV in the subject. In some embodiments, the sense sequence and the antisense sequence of the first siRNA and of the second siRNA, respectively, have a two T overhang on said 3′ ends. In some embodiments, the sense sequence, with overhang, of the first siRNA consists of a sequence selected from the group consisting of SEQ ID NO:11, SEQ ID NO:15, SEQ ID NO:17, SEQ ID NO:19, and SEQ ID NO:23, provided that if the sense sequence selected is SEQ ID NO:17, the sense sequence selected for the sense sequence of the second siRNA is not SEQ ID NO:19. In some embodiments, the sense sequence, with overhang, of said second siRNA consists of a sequence selected from the group consisting of SEQ ID NO:11, SEQ ID NO:15, SEQ ID NO:17, SEQ ID NO:19, and SEQ ID NO:23. In some embodiments, the sense and antisense sequences of said first siRNA have the sequences SEQ ID NO:17 and SEQ ID NO:18, respectively, and the sense and antisense sequences of the antisense sequences of said second siRNA have the sequences of SEQ ID NO:23 and SEQ ID NO:24, respectively. In some embodiments, the first siRNA and the second siRNA are complexed to or encapsulated in a nanosome, thereby forming an siRNA-nanosome. In some embodiments, the nanosome comprises a cationic lipid. In some embodiments, the cationic lipid is selected from the group consisting of 3β-[N-(N′,N′-dimethylaminoethane)-carbamoyl]cholesterol hydrochloride (DC-cholesterol); 1,2-dioleoyl-3-trimethylammonium-propane (DOTAP); 1,2-dioleoyl-3-dimethylammonium-propane (DODAP); dimethyldioctadecylammonium (DDAB); 1,2-dilauroyl-sn-glycero-3-ethylphosphocholine (Ethyl-PC), or 1,2-di-O-octadecenyl-3-trimethylammonium propane (DOTMA). In some embodiments, the cationic lipid is DOTAP. In some embodiments, the nanosome further comprises a neutral lipid. In some embodiments, the neutral lipid is cholesterol. In some embodiments, the first and the second siRNAs are condensed with a cationic polymer. In some embodiments, the cationic polymer is protamine sulfate. In some embodiments, the nanosome comprises a sugar. In some embodiments, the sugar is selected from the group consisting of trehalose, sucrose, lactose and mannitol. In some embodiments, the sugar is trehalose. In some embodiments, the the siRNA-nanosome is a rehydrated powder, a rehydrated lyophilized powder, or a rehydrated spray dried powder. In some embodiments, the siRNA-nanosomes are sonicated within approximately 1½ hours prior to administration to the subject. In some embodiments, the siRNA-nanosomes are sonicated within one hour prior to administration to the subject. In some embodiments, the siRNA-nanosomes are sonicated 5 to approximately 45 minutes before administration to the subject. In some embodiments, the sonication is for less than 5 minutes. In some embodiments, the sonication is for less than 3 minutes. In some embodiments, the HCV is selected from HCV genotypes type 1a, 1b, and 2a. In some embodiments, the HCV is of genotype is 2a.

In another group of embodiments, the invention provides methods of preparing a nucleic acid agent for administration to the liver of a subject in need thereof. The methods comprise (a) providing a plurality of the nucleic acid agent complexed to or encapsulated in nanosomes, the nanosomes having an average size, and (b) sonicating the plurality of the nucleic acid agent complexed to or encapsulated in the nanosome to reduce the average size of the nanosomes, thereby preparing the nucleic acid agent for administration. In some embodiments, the nucleic acid agent is an siRNA. In some embodiments, the nucleic acid agent is an expression plasmid. In some embodiments, the sonication is for less than 5 minutes. In some embodiments, the sonication is conducted within 1.5 hours of administration of the nanosomes to the subject. In some embodiments, the sonication is conducted 5-45 before administration of the nanosomes to the subject. In some embodiments, the sonication is conducted immediately before administration of the nanosomes to the subject. In some embodiments, the average size of a majority of said nanosomes is 150 nm or less.

### BRIEF DESCRIPTION OF THE DRAWINGS

FIGS. 1A-D. FIGS. 1A-1 and 1A-2 shows a proposed structure for the highly conserved 5′UTR of the HCV RNA genome and the positions of siRNA targets selected within the 5′UTR, designated by the abbreviation “si” followed by a number denoting the position of the nucleotide of the sequence targeted by the siRNA. (Note: the structure of the 5′UTR was separated into FIGS. 1A-1 and 1A-2 due to size constraints, but presents one continuous structure of contiguous nucleotides, SEQ ID NO:34). The Roman numerals and letters designate the secondary structure formed by the 5′UTR. FIG. 2. FIG. 2 shows the sequence of the 5′UTR of HCV genotype 2a (SEQ ID NO:35). The locations of 13 different siRNAs in the 5′UTR of the HCV genome along with seed match sites of miR122 are shown. FIG. 1C. FIG. 1C is a graph showing cell viability (in %, vertical axis) due to the addition of siRNA-nanosome complex, as measured by MTT assay. Doxorubicin (ng/ml) was used as a positive control (Doxo). Blank nanosome (“NP”) and siRNA-nanosomes (“siRNA+NP”) were used in the pmole concentrations shown along the horizontal axis. FIG. 1D. FIG. 1D is a graph showing activation of the endogenous IFN-α promoter in Huh-7.5 cells due to delivery of siRNA nanosome, as assessed by measuring the ISRE-luciferase activity. Equal numbers (4×10⁴ cells/well) of Huh-7.5 cells were seeded and the next day 0.5 μg of pISRE-Firefly luciferase reporter plasmid was transfected by FuGENE-6. Two hours after this transfection, siRNA-nanosome complex was added. Twenty four hours later, luciferase activity was measured. Each experiment in FIGS. 1C and 1D was performed in triplicate; the bars represents the standard deviations.

FIGS. 2A-B. Both Figures: R4-GFP cells (cells stably transfected with viral replicon RNA that expresses green fluorescent protein, GFP) were seeded in wells (2×10⁵ cells/well). The next day, 30 μl of siRNA-nanosome complex (100 pmole) was added drop wise to the cell culture and mixed thoroughly. After 72 hours, HCV-GFP expression was monitored under a fluorescent microscope. FIG. 2A presents graphs showing flow cytometric analyses of the R4-GFP cells, quantified to show the GFP positive cells as a percentage. In untreated cells, most of the cells are glowing, showing unimpeded viral replication. A shift of the peak to the left indicates a reduction in the number of cells in which virus is replicating. FIG. 2B. FIG. 2B is a graph showing the concentration-dependent antiviral effect of siRNA. The reduction of HCV-GFP expression was quantified by flow cytometry after two consecutive treatments with three concentrations of the indicated siRNA, complexed or encapsulated in nanosomes as described in the Examples, within a 5 days interval. Experiments were performed in triplicate; the bar represents the standard deviation.

FIGS. 3A-F. FIGS. 3A-F show the inhibition of HCV replication by combination siRNA-treatment in a replicon cell line. R4-GFP replicon cells (2×10⁵/well) were treated with either a single siRNA (complexed to nanosomes) or a combination of two siRNA (complexed to nanosomes). The cells were then cultured in media supplemented with G-418 for 60-days. During the sixty day period, the cultures were repeatedly treated with siRNA. The first five treatments (T1-T5) were given at 5 day intervals, followed by 5 more treatments (T6-T10) at 7 day intervals. FIG. 3A. FIG. 3A is a photograph showing the number of G-418 resistant cell colonies following siRNA treatment. In this standard assay to study full-cycle replication of HCV, the number of G-418 resistant cell colonies is directly proportional to the HCV replication. HCV that survived siRNA treatment due to virus escape mechanisms develops G-418 resistant cell colonies. A smaller number of colonies means the siRNA treatment had a strong anti-viral effect; a larger number of colonies means the siRNA had a less potent antiviral effect. FIG. 3B. FIG. 3B is a graph showing the HCV RNA levels in cells treated by si321, by si359, by co-administration of both siRNAs, or by mock treatment, as measured by RT-qPCR. Representative data (mean±SD) from at least 3 independent experiments are shown. FIG. 3C. FIG. 3C shows an escape mutant analysis of replicon clones surviving long-term treatment with a single siRNA by sequence analysis. The nucleotide substitutions seen in the escape mutant clones are shown for comparison. S=Samples. C=Clone number. SC=Sub-clone number. M=Mock (R4-GFP cells). si321R, si359R indicates cell lines resistant to the stated siRNA. FIG. 3D. FIG. 3D is a photograph showing complete clearance of HCV replication from replicon cultures after 3 treatments using combinations of si321+si359. The presence of residual intracellular HCV replicon RNA in the culture over 60 days was assayed by the appearance of G-418 resistant cell colonies in the presence and absence of siRNAs treatment. FIG. 3E. FIG. 3E is a graph showing the HCV RNA level after three of the indicated treatments, as determined by RT-qPCR over 60 days. Representative data (mean±SD) from at least 3 independent experiments are shown. The dotted line indicates the detection limit of the assay. FIG. 3F. FIG. 3F are photographs showing (i) ethidium bromide staining of RT-nested PCR products of cultured cells treated with two siRNAs in combination, (ii) corresponding Southern blot analysis at day 25, 39 and 60 after receiving the third siRNA treatment, and (iii). PCR amplification of GAPDH used as an internal control. The total RNA from Mock and siIRR treated cells remained HCV positive.

FIGS. 4A-E. All Figures are studies of inhibition of HCV replication in an infectious cell culture model by combination of two siRNA treatments. Huh-7.5 cells (1.2×10⁵ cells/well) were infected with JFH1-ΔV3-Rluc virus with a MOI of 0.1 overnight. After 24 hours, cells were treated with siRNA-nanosome complex. FIG. 4A is a graph showing the antiviral effect of 13 different siRNA-nanosomes determined by measuring Renilla luciferase activity. FIG. 4B. FIG. 4B is a graph showing the results of studies in which infected cells were treated with either single (100 pmole) or combined siRNAs (50 and 100 pmole each). Five 5 days post-treatment, half of the treated cells were used for a second round of siRNA treatment. After two consecutive treatments (T1 and T2), cells were cultured up to 30 days without treatment. HCV replication in the siRNA-treated infected cultures was determined by measuring Renilla luciferase activity. FIG. 4C. FIG. 4C is a graph showing intracellular HCV RNA levels in siRNA-treated cultures as determined by RT-qPCR. The results are compared to untreated controls and expressed as a log copies per micro gram of cellular RNA. Mean±standard deviations from a representative experiment performed in triplicate are shown. The dotted line indicates the detection limit of the assay. FIG. 4D. FIG. 4D is a photograph confirming the antiviral effect of siRNA treated infected culture by the detection of HCV core protein by Western blot analysis. Levels of beta-actin assured equal amounts of protein were loaded in each well. “P” indicates the number of passages of the infected culture. FIG. 4E. FIG. 4E shows photographs confirming the sustained antiviral effect of single and combination siRNA treatment using persistently HCV infected Huh-7.5 cells in the culture by core immunostaining. Persistently infected Huh-7.5 cells in culture were repeatedly treated with 100 pmole of single or combination of two siRNA (si321 and si359) at a five day intervals. Persistently infected cells treated with irrelevant siRNA targeted to Epstein Barr virus (EBNA1) were used as a control. The success of single versus combination siRNA treatment clearing the HCV replication in the infected culture was examined after each treatment by examining the number of cells expressing core protein.

FIGS. 5A-E. FIGS. 5A-E show the antiviral effect of siRNA-nanosome treatment in a subcutaneous (“S/C”) tumor model. FIG. 5A. FIG. 5A shows siRNA delivery efficiency in the tumor after peritumoral injection of Cy3-labeled si321-nanosome by fluorescence microscopy. FIG. 5B. FIG. 5B (i) is a photograph showing the development of S/C tumors in untreated mice (“Mock”) and in mice treated with a combination of siRNA321 and si359. FIG. 5B (ii) is a photograph showing GFP expression under fluorescence microscopy of frozen section of S/C tumors in untreated (Mock) and siRNA (si321+si359) treated mice. FIG. 5C is a photograph of colony assays showing the replication of HCV sub-genomic RNA in tumor cells isolated from S/C tumors of Mock, control siRNA (siIRR) and HCV specific siRNA (si321+si359) treated mice. FIG. 5D. FIG. 5D sets forth photographs showing detection of HCV positive strand RNA by RPA in untreated (“Mock”) mice or in mice treated with an irrelevant siRNA (“siIRR”, targeted to Epstein-Barr virus nuclear antigen) or with a combination of si321 and si359. GAPDH was used as a control. FIG. 5E. FIG. 5E is a graph showing HCV RNA levels as measured by RT-qPCR. The dotted line indicates the detection limit of the assay.

FIGS. 6A-E. FIGS. 6A-e show the antiviral effect of combination siRNA-nanosome treatment in a liver tumor model. FIG. 6A is a graph showing the body weight of SCID/NOD mice either mock treated (“Mock”) or treated with a combination of siRNAs. FIG. 6B. Five each of untreated (Mock) and siRNA-nanosome treated (si321+si359) mice had intrahepatic HCC of liver tissue sections examined by hematoxylin and eosin (H&E) staining. The upper five panels of FIG. 6B are photographs showing the H&E staining of the frozen liver of the Mock treated mice. The lower five panels of FIG. 6B show the H&E staining of the frozen liver of the siRNA-nanosome treated mice. The arrows indicate the presence of HCC in the liver. FIG. 6C. FIG. 6C sets forth photographs showing the replication of HCV sub-genomic RNA in liver tumors of mice either not treated (“Mock”), treated with an irrelevant siRNA as a control (“siIRR,” targeted to Epstein-Barr virus nuclear antigen), or with a combination of si321+si359 by colony assay under G-418 (1mg/ml) selection for 3 weeks. FIG. 6D. FIG. 6D are panels showing detection of HCV positive strand RNA by RPA. GAPDH was used as a control. Mock=untreated mice, as a control. siIRR=mice treated with an irrelevant siRNA (targeted to Epstein-Barr virus nuclear antigen) as a second control. si321+si359=mice treated with a combination of the stated siRNA-nanosomes. FIG. 6E. FIG. 6E is a graph showing HCV-RNA levels in the livers of untreated mice (Mock), mice treated with an irrelevant siRNA (siIRR), and mice treated with a combination of the stated siRNA-nanosomes.

FIGS. 7A-E. All Figures: nanosomes containing control siRNA (EBNA1) were freshly prepared and 100 μl of the siRNA-nanosome at a dose of 5 mg/Kg body weight was slowly infused to BALB/c mice through tail vein. Mice were injected with 100 μl of either siRNA-nanosome or saline every other day for seven days. A total of 35 mice were divided into 7 groups and were used as untreated, saline-treated (4, 24 and 7-days) and siRNA-nanosome treated (4, 24 and 7-days) groups. FIG. 7A is a graph showing the body weights of 10 mice that received multiple injections of saline or siRNA-nanosome (on days shown by arrows) over a period of 8 days. FIGS. 7B and 7C. For FIGS. 7B and 7C, blood samples were collected at the indicated time points and serum levels of ALT and AST levels in mice were measured after systemic administration of siRNA-nanosome or saline. FIG. 7B is a graph of the levels of ALT in untreated mice, and in mice at 4 hours, 24 hours and 7 days after siRNA-nanosome treatment. FIG. 7C is a graph of the levels of AST in untreated mice, and in mice at 4 hours, 24 hours and 7 days after siRNA-nanosome treatment. Both Figures: values are expressed as mean+SD. The differences of AST and ALT values between the untreated, control and siRNA-nanosome treated were not statistically significant (NS). FIG. 7D. FIG. 7D presents photographs of formalin-fixed, hematoxylin and eosin-stained tissue sections of heart, lung, liver, spleen and kidney of untreated mice, and of mice 24 hours after siRNA delivery. No evidence of toxicity is visible. Photos taken 7 days after siRNA treatment likewise showed no toxicity. FIG. 7E. FIG. 7E presents photographs of the HCC and surrounding non-tumor areas of the liver of untreated control mice and of mice treated with siRNAs. No evidence is seen of liver cell toxicity after siRNA-nanosome administration. The arrows indicate the presence of HCC in the liver.

FIG. 8. FIG. 8 presents six graphs showing the effect on HCV replication of a series of siRNAs administered individually or in various combinations, at three different concentrations, in pmol. The cells are stably transfected with HCV bearing a sequence for GFP. The flow cytometric analyses quantifies to show the GFP-positive cells as a percentage. In untreated cells, over 80% of the cells fluoresce (are GFP-positive), showing uninhibited viral replication. The vertical axis shows the percentage of GFP-positive cells following the treatment shown on the horizontal axis. A shorter bar on the graph shows that the indicated treatment inhibited HCV replication. Mock=untreated cells. “IFN+” =cells treated with interferon. siIRR=cells treated with irrelevant siRNA (targeted to Epstein-Barr virus nuclear antigen) as a control. Horizontal black marks above an siRNA or combination of siRNAs indicates the administration to the cells of that siRNA or combination of siRNAs at the concentrations, in pmol, shown above the black mark.

FIG. 9. FIG. 9 is a graph showing the effect of two different siRNA combinations on inhibiting HCV replication in cells stably transfected with a Renilla luciferase reporter-based full-length HCV clone (JFH-AV3-Rluc). Cultures were followed for 80 days. Arrows indicate the days on which the indicated combinations of siRNAs-nanosomes were administered to cells (for those cells treated.) “Untreated” designates transfected cells that were not treated. “Cells only” designates cells that were not transfected, as a control. After 80 days, cells treated with a combination of si321 and si359 showed Renilla activity virtually indistinguishable from cells not infected with HCV, whereas cells treated with a combination of si333 and si351 showed an initial decrease in viral replication, followed by a rebound.

### DETAILED DESCRIPTION

### Introduction

As noted in the Background, hepatitis C virus, or “HCV” infection affects millions of persons in the U.S. and around the world, and chronic HCV infection causes cirrhosis and liver cancer. Unfortunately, current options for inhibiting HCV replication are limited.

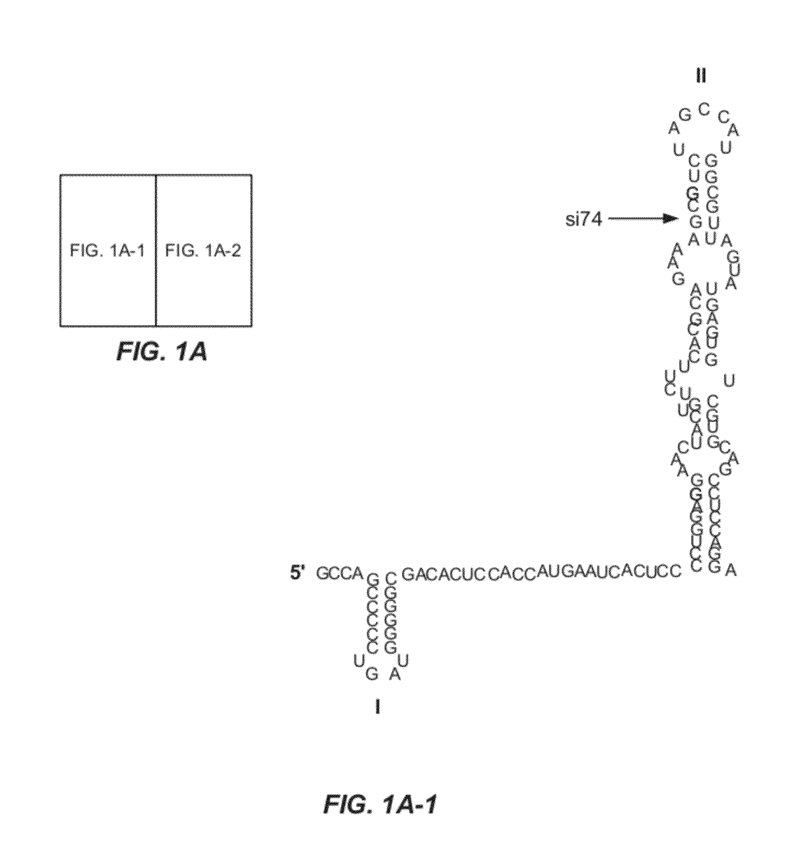

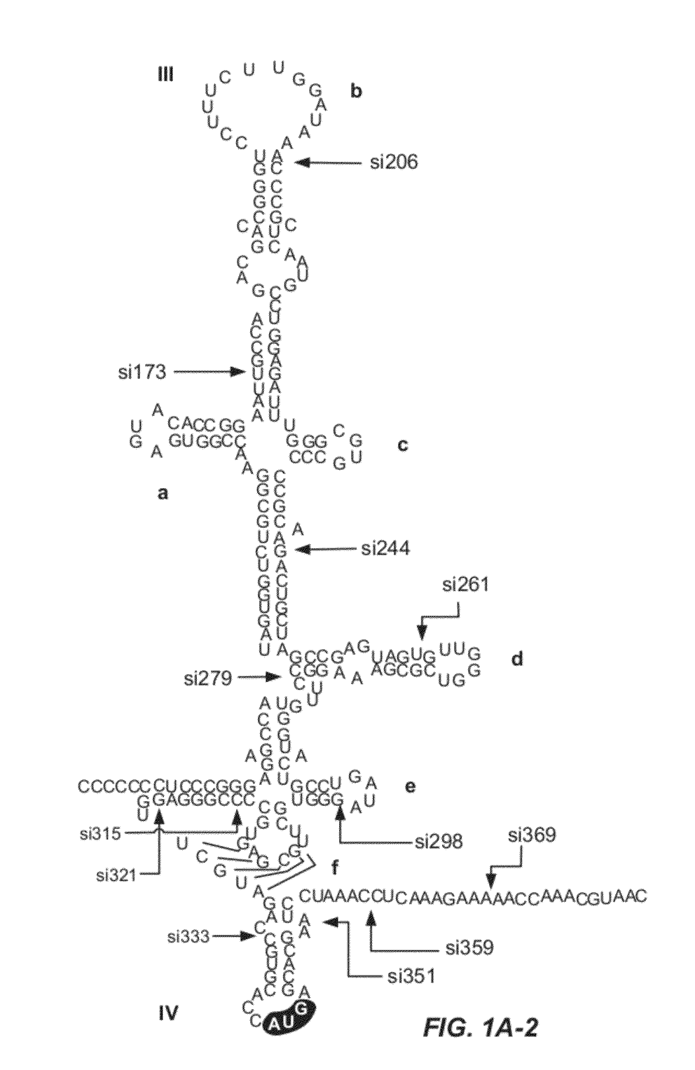

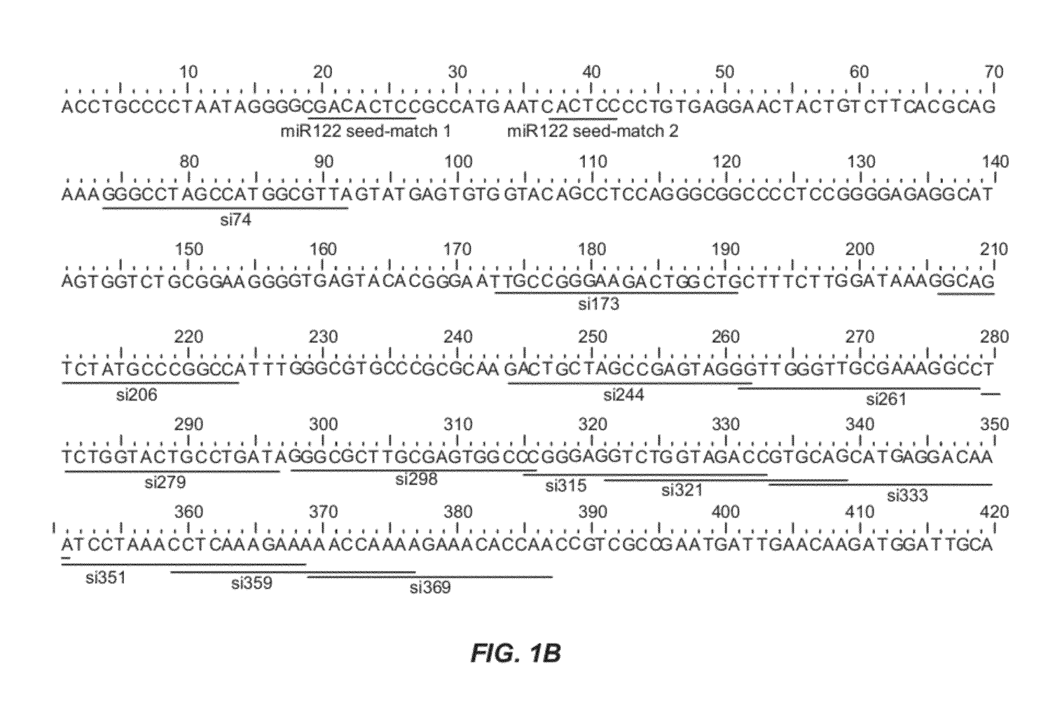

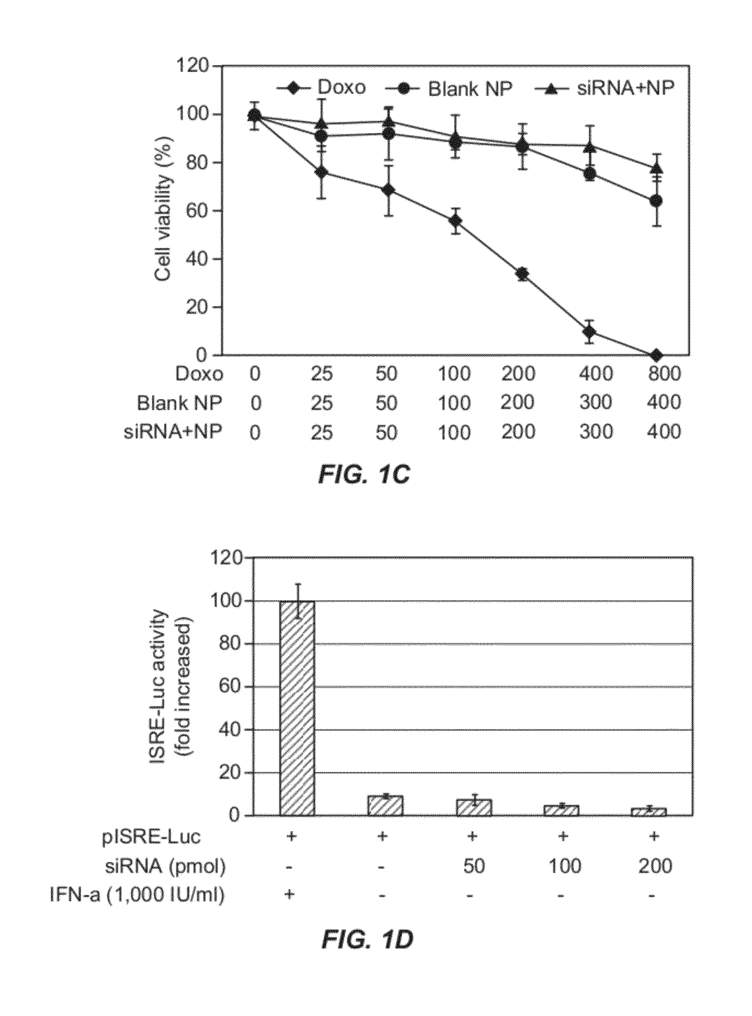

For some time, HCV has been considered a promising target for nucleic acid based approaches to inhibit viral replication. In particular, HCV's 5′ untranslated region (“5′UTR”) has been investigated as a target for siRNAs to inhibit HCV. Unfortunately, as shown in FIG. 1A, the HCV 5′UTR has a complicated secondary structure which renders it difficult to predict whether any particular siRNA will be successful in inhibiting viral replication. Further, studies herein demonstrate that the administration of multiple copies of a single siRNA is likely to be ine ffective, as the virus develops variations that permit it to continue to replicate. Moreover, the studies reported herein show that administration of two different siRNAs in some cases does not inhibit viral replication more than one of the siRNAs administered alone. Further, even if one has found a promising siRNA, delivering it to cells has proved difficult.

The present invention provides significant and surprising solutions to these problems. Surprisingly, despite the difficulties noted above, the studies reported herein report the invention of particular combinations of siRNAs that strongly inhibit HCV replication both in cell culture and in vivo. Further surprisingly, the combinations can induce long term inhibition of HCV replication. Almost two months after treatment was ceased with one combination of siRNAs, viral activity in the treated cells was almost indistinguishable from levels in cells that had never been infected. The studies underlying this aspect of the invention also showed that only half the tested combinations of siRNA resulted in strong inhibition of HCV replication. Indeed, some combinations of siRNA did not result in inhibition of HCV replication as strong as the inhibition induced by the better siRNA of the combination when administered by itself. In this regard, the results show that combinations of siRNA in which the siRNA are to overlapping portions of the HCV 5′UTR sequence do not have synergistic or even additive effects in inhibiting HCV replication. Accordingly, the results show that to enhance HCV inhibition, the combinations of siRNAs should target non-overlapping portions of the 5′UTR. The combination of two or more siRNAs can be co-administered either as separate agents (for example, by intravenous injection of the first siRNA, followed shortly by intravenous injection of the second siRNA) or, preferably, by administration together in a single composition, such as in a solution for intravenous injection, which solution comprises both siRNAs.

As also noted in the Background, the administration of siRNAs to the liver cells that are specifically infected by HCV has proved problematic as well. The studies underlying the present invention demonstrate that condensing the siRNAs and complexing or encapsulating the condensed siRNAs with nanoparticles of a cationic lipid and cholesterol, and in particular, reducing the average size of the nanoparticles below 150 nm, increases the ability of the nanoparticles to reach infected cells. Our studies indicated that the previous methods for producing nanosomes or other nanoparticles, such as high pressure homogenization (see, Kundu et al., J Pharm Pharmacol 62(9):1103-11 (2010)), resulted in populations of nanoparticles substantial percentages of which were too large to cross liver fenestrae. Decreasing the average size of the nanoparticles would increase the percentage able to enter liver cells and permit the practitioner to achieve the same therapeutic effect with a smaller dose. Since the effects of administering nucleic acid-nanosome formulations are, like those of most agents, in part dose-related, the ability to achieve the same efficacy with a smaller dose would be desirable. It therefore would be desirable to reduce the average size of the nanosomes below that of the nanosomes formed by conventional techniques.

Surprisingly, the studies reported here show we were able to reduce the average size of the particles by a simple technique without reducing their efficacy for transfecting cells. The studies reported below show that sonicating the nanoparticles for as little as 30 seconds after the siRNA has been complexed with or encapsulated within the nanoparticles reduced the average size of the particles and increased the percentage below the 150 nm threshold. While sonication has been used by others to reduce aggregation of nanoparticles prior to adding siRNA or other nucleic acid agents, it has not to our knowledge been used after the siRNA or other nucleic acid agent was complexed with or encapsulated in the nanoparticle. It could not be predicted prior to attempting it whether sonication would cause some or all of the siRNA to dissociate from the nanoparticle and render them ineffective for their intended purpose. Further, it could not be predicted prior to attempting it whether the siRNA would be broken up or otherwise lose its ability to interfere with HCV replication. The studies reported below show that the siRNA remained complexed or encapsulated after sonication, and that the siRNA remained effective in interfering with HCV replication in transfected cells. Since the improved ability to deliver nucleic acids by this method is not limited to siRNAs, the method is expected to be useful for any condition for which delivery of nucleic acid agents to the liver would be useful, including, but not limited to, hepatitis A, hepatitis B, liver cancer (including hepatocellular carcinoma, cholangiocarcinoma, angiosarcoma and hemangiosarcoma of the liver), hereditary diseases such as hemochromatosis, Wilson's disease transthyretin-related hereditary amyloidosis, Gilbert's syndrome, or alpha-1 antitrypsin deficiency.

Systemic administration of siRNA-nanoparticles in a mouse model caused no apparent liver damage to the mice or other damage evident on histological examination of multiple organs. Further, systemic injections of combinations of siRNA complexed or encapsulated in nanoparticles into mice bearing xenografts of human liver tumors resulted in significant reductions in HCV replication. Accordingly, the siRNAs of the invention, particularly when administered complexed or encapsulated in nanoparticles, and particularly when those nanoparticles have an average size of 150 nm or less, are expected to be useful in inhibiting HCV replication in subjects in need thereof.

The siRNAs tested in the studies underlying the present invention were derived from the sequence of HCV2a. The 5′UTR of HCV genotypes is conserved, and it is expected that the inventive siRNAs will be useful in inhibiting replication of genotypes other than 2a. In particular, it is expected that they will be useful in inhibiting replication of genotype 1 a, the most common HCV genotype found in individuals in the U.S. It is also expected that they will be useful in inhibiting replication of HCV genotype 1b, the second most common HCV found in individuals in the U.S. The sequences targeted by siRNAs si321 and si359, for example, are the same in all three genotypes, 1a, 1b and 2a and the siRNAs should be equally effective in inhibiting replication of all three genotypes. Due to the conservation of the 5′UTR, the siRNAs are expected to be effective in inhibiting replication of HCVs of other genotypes as well.

If desired, the practitioner can genotype the particular virus infecting his or her patient to confirm whether the inventive compositions and methods would be useful in reducing HCV replication in the patient. A variety of techniques for genotyping HCV are known in the art, including several versions of real-time PCR, and restriction fragment length polymorphism. Methods of performing genotyping by these methods is taught in, for example: Davidson et al., J Genl Virol 76(Pt 5):1197-1204 (1995); Murphy et al., J Clin Microbiol 45(4):1102-1112 (2007); Cantaloube et al., J Clin Microbiol 44(6):2051-2056 (2006); Lindh and Hannoun, J Clin Virol 34(2):108-114 (2005); and Rolfe et al., J Clin Virol 34(2):115-121 (2005).

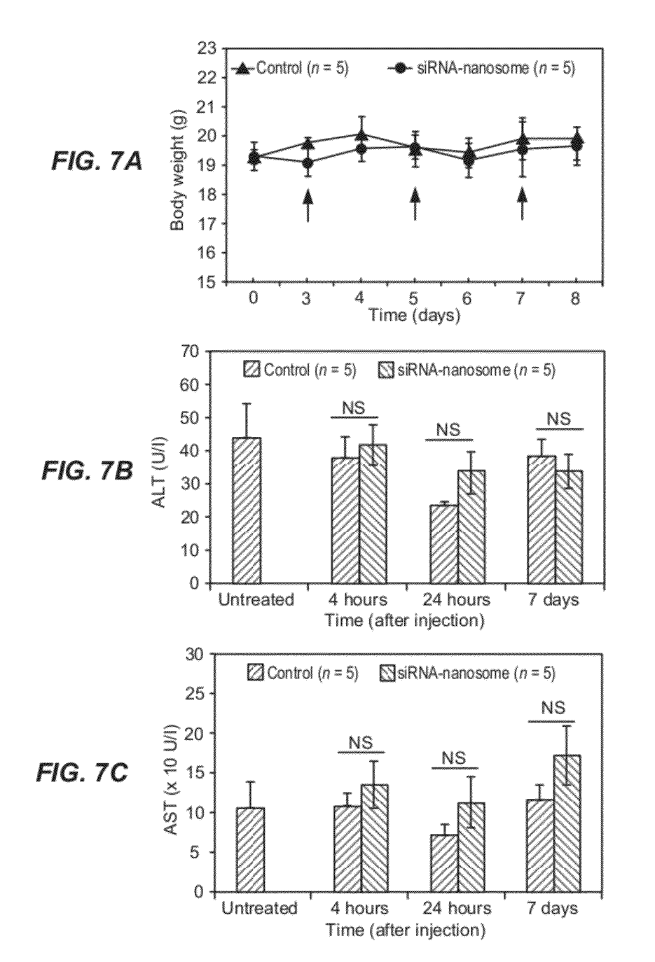

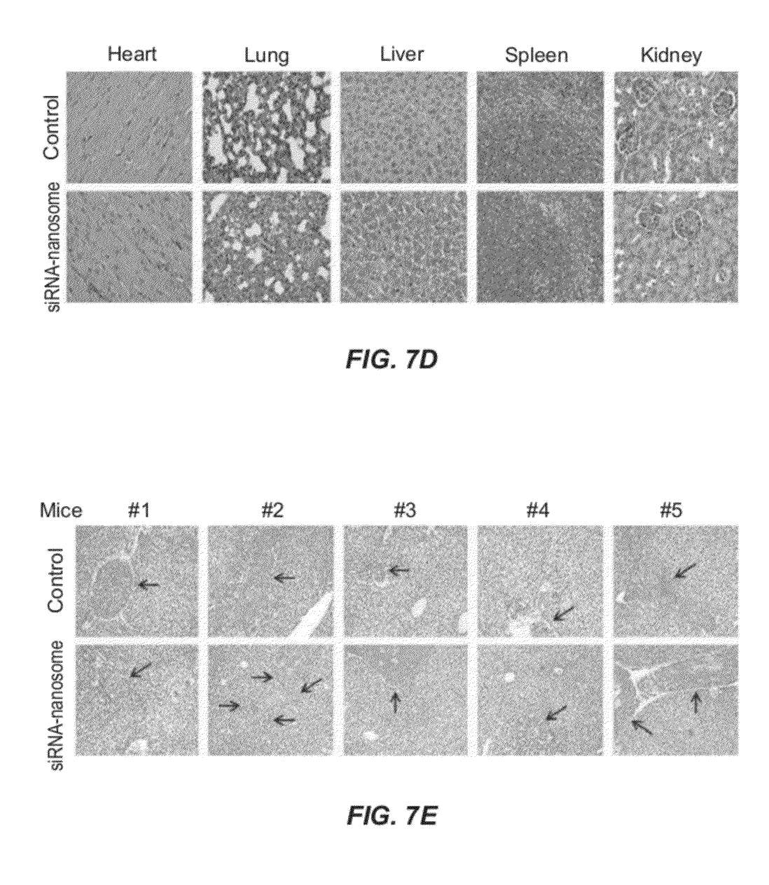

One concern that has hindered the use of siRNA therapeutics has been the occurrence of so-called “off-target effects,” in which the siRNA silences expression of a gene other than the intended target. A NCBI blast homology search (BLASTIN 2.2.26+) was performed on the sequences of the siRNAs set forth in Table 1. There were no 100% matches with any human genomic sequence. It should also be noted that these siRNAs were well tolerated in a mouse model, with no behavioral abnormalities noted in the animals before sacrifice, and no damage evident in the cells of organs examined following administration of the siRNAs (see, e.g., FIG. 7D).

### Definitions

Units, prefixes, and symbols are denoted in their Systeme International de Unites (SI) accepted form. Numeric ranges are inclusive of the numbers defining the range. Unless otherwise indicated, nucleic acids are written left to right in 5′ to 3′ orientation. The headings provided herein are not limitations of the various aspects or embodiments of the invention, which can be had by reference to the specification as a whole. Accordingly, the terms defined immediately below are more fully defined by reference to the specification in its entirety.”

As used herein, “nanosome” refers to nano-sized particles of a cationic lipid and a non-toxic neutral lipid that can serve as a carrier of nucleic acids. The lipids of such nanoparticles may form a bilayer or have an internal compartment but, following practice in the art, will be referred to herein as “nanosomes” regardless of whether the lipids do form a bilayer or form an internal compartment.

The phrase “inhibiting the replication of hepatitis C virus” in a subject means inhibiting replication of the virus by at least 10%, more preferably 20%, still more preferably 20%, even more preferably 30%, yet more preferably 40%, more preferably 50%, more preferably 60%, more preferably 70%, still more preferably 80%, yet more preferably 90%, preferably 90-99%, and most preferably 100% Inhibition of replication of hepatitis C virus (HCV) is typically measured in the art by the number of viruses or of “viral equivalents,” per milliliter of blood, as determined by a quantitative HCV RNA PCR test or the more sensitive (and preferred, for this purpose) qualitative HCV RNA test.

“Co-administration” and “combination therapy” are used herein as synonyms and refer to the administrating to a subject two or more small interfering RNAs (“siRNAs”) or other therapeutic agents close enough in time so that therapeutically effective amounts of each of the siRNAs or agents are present at the subject at the same time. The siRNAs or other therapeutic agents can be administered by the same route or by different routes. For example, a first siRNA of the combination selected may be administered by intravenous injection while a second siRNA of the combination may be administered into the portal vein. Alternatively, for example, all the siRNAs may be administered by intravenous injection. In preferred embodiments, the two or more siRNAs are administered in a single composition, such as in a solution for intravenous injection in which the two or more siRNAs are present.

The terms “double-stranded RNA” or “dsRNA”, refer to a complex of ribonucleic acid molecules having a duplex structure comprising two anti-parallel and substantially complementary nucleic acid strands. The two strands forming the duplex structure may be different portions of one larger RNA molecule, or they may be separate RNA molecules. Where the RNA molecules are separate molecules, such dsRNA are often referred to as siRNA (“short interfering RNA”). The RNA strands may have the same or a different number of nucleotides. The maximum number of base pairs is the number of nucleotides in the shortest strand of the dsRNA minus any overhangs that are present in the duplex. In addition to the duplex structure, a dsRNA may comprise one or more nucleotide overhangs.

The studies underlying the invention used siRNAs targeted to particular portions of the 5′ untranslated region (“5′UTR”) of the hepatitis C virus (“HCV”). Each of the siRNAs studied has a 19-nucleotide (“nt”) sequence corresponding to a target sequence within the sequence of the HCV 5′UTR shown in FIG. 1B, and is numbered according to the number of the first nucleotide of the target sequence. The siRNAs studied further have a two T overhang on the 3′ end of each 19-nt sequence. Thus, for example, “si279” refers to an siRNA consisting of (a) a first sequence of 19 nucleotides corresponding to 19 nucleotides of the HCV 5′UTR sequence shown in FIG. 1B starting at position 279 of FIG. 1B, plus (b) a two T overhang and a second sequence, complementary to the sequence of (a), above, plus a two T overhang on the 3′ end of that sequence. The particular siRNAs used in the studies underlying the invention are set forth in Table 1, below.

With respect to compositions of siRNAs, the term “isolated” refers to dsRNAs or siRNAs that have been synthesized as individual entities, rather than being incidentally created by cleavage from a larger dsRNA by an endoribonuclease, such as Dicer.

As used herein, the phrase “nucleic acid agent” refers to a nucleic acid sequence intended to have a desired therapeutic effect when delivered to cells of a subject in need thereof, and in particular, to liver cells. The nucleic acid agent may be, for example, a nucleic acid, such as an siRNA, that induces RNA interference that silences expression of a target sequence, or an expression plasmid or expression cassette which, when introduced into cells, results in expression of a desired protein.

A “nucleotide overhang” refers to any unpaired nucleotide or nucleotides that protrude from the duplex structure of a dsRNA when a 3′-end of one strand of a dsRNA, such as a siRNA, extends beyond the 5′-end of the other strand, or vice versa.

Persons of skill will be aware that where the desired nucleic acid molecule is RNA, a T (thymine) in a genomic sequence would be substituted with U (uracil). Overhangs in a dsRNA, such as an siRNA, however, are typically composed of two Ts.

A “blunt end” means that there are no unpaired nucleotides at that end of a dsRNA, such as a siRNA, i.e., there is no nucleotide overhang. A “blunt ended” dsRNA is a dsRNA that is double-stranded over its entire length, i.e., there is no nucleotide overhang at either end of the molecule.

The term “antisense strand” refers to the strand of a dsRNA, such as a siRNA, which includes a region that is substantially complementary to a target sequence. This strand is also known as the “guide” sequence, and is used in the functioning RNA-induced silencing complex, or “RISC,” to guide the complex to the correct mRNA for cleavage. As used herein, the term “region of complementarity” refers to the region on the antisense strand that is substantially complementary to a sequence, for example a target sequence, as defined herein. Where the region of complementarity is not fully complementary to the target sequence, the mismatches are most tolerated in the terminal regions and, if present, are generally in a terminal region or regions, e.g., within 4, 3, 2, or 1 nucleotides of the 5′ and/or 3′ terminus This use of “antisense,” because it relates to an RNA compound, is different from its use with respect to so-called “antisense” DNA compounds, which relate to a different type of nucleic acid therapeutic.

The term “sense strand,” as used herein, refers to the strand of a dsRNA, such as a siRNA, that includes a region that is complementary to a region of the antisense strand. This strand is also known as the “anti-guide” sequence because it contains the same sequence of nucleotides as the target sequence and therefore binds specifically to the guide sequence. The top strand listed for each siRNA set forth in Table 1 may be considered the sense strand.

“Introducing into a cell”, when referring to a siRNA or nucleic acid agent, means facilitating uptake or absorption into the cell, as is understood by those skilled in the art. Absorption or uptake of dsRNA can occur through unaided diffusive or active cellular processes, or by auxiliary agents or devices. The meaning of this term is not limited to cells in vitro; a siRNA or other nucleic acid agent may also be “introduced into a cell”, wherein the cell is part of a living organism. In such instance, introduction into the cell will include the delivery to the organism. For example, for in vivo delivery, a siRNA or other nucleic acid agent can be injected into a tissue site or administered systemically.

The term “nanoparticle” can technically be used to refer to any composition of 999 nm or smaller in size. It is more particularly used herein to refer to particles comprising cationic lipids and neutral lipids that can be used as carriers to deliver nucleic acids to cells.

The terms “individual,” “subject,” and “patient” refer to a mammal, including, but not limited to, non-human primates (e.g., simians), and humans.

### Hepatitis C Virus

Hepatitis C Virus (HCV) is a small (40 to 60 nanometers in diameter), enveloped, single-stranded RNA virus of the family Flaviviridae and genus hepacivirus. The genome is approximately 10,000 nucleotides and encodes a single polyprotein of about 3,000 amino acids, which is post-transcriptionally cleaved into 10 polypeptides, including 3 major structural (C, E1, and E2) and multiple non-structural proteins ([NS] NS2 to NS5). The NS proteins include enzymes necessary for protein processing (proteases) and viral replication (RNA polymerase). Because the virus mutates rapidly, changes in the envelope proteins may help it evade the immune system.

Hepatitis C virus (HCV) is a small, enveloped, member of the family Flaviviridae . It is a positive-strand RNA virus with a single oligoribonucleotide genomic sequence of about 9600 bases which encodes a polyprotein of about 3,010 amino acids. HCV genomic RNA includes a 5′-nontranslated region (5′UTR) that includes an Internal Ribosome Entry Site (IRES) that contributes to translation of the polyprotein. The protein products of the HCV gene consist of the structural proteins C, E1, and E2, and the non-structural proteins NS2, NS3, NS4A and NS4B, and NS5A and NS5B. The nonstructural (NS) proteins are believed to provide the catalytic machinery for viral replication. The NS3 protease releases NS5B, the RNA-dependent RNA polymerase from the polyprotein chain. HCV NS5B polymerase is required for the synthesis of a double-stranded RNA from a single-stranded viral RNA that serves as a template in the replication cycle of HCV.

According to the website of the Centers for Disease Control and Prevention, HCV is the most common chronic bloodborne infection in the United States. While about 15% of persons infected with HCV are able to clear the virus on their own, the remainder become chronically infected. The CDC estimates 3.2 million people are chronically infected with HCV in the United States. Most are asymptomatic, but many have chronic liver disease. Laboratory criteria for diagnosis include detection of anti-HCV antibodies by solid-phase enzyme immunoassay (EIA), confirmed by a more specific assay such as nucleic acid testing or a HCV recombinant imunoblot assay (RIBA).

There are at least 6 major HCV genotypes, designated by the numbers 1-6, and more than 90 subtypes of HCV, generally designated by letters. The different genotypes have different geographic distributions. Genotypes la and lb are the most common in the United States, with genotype 1a responsible for about 57% of infections and lb occurring in about 17%. Of infections. Genotypes 2a and 2b are responsible for approximately 15% of U.S. HCV infections), with genotype 3 responsible for approximately 7%. Genotype 2a is the most common genotype in China and Japan. Patients with genotypes 2 and 3 are more likely to respond to interferon treatment.

As stated in U.S. Published Patent Application 20120035240, the main goal of treatment of chronic hepatitis C is to eliminate detectable viral RNA from the blood. Lack of detectable hepatitis C virus RNA from blood six months after completing therapy is known as a sustained response. Studies suggest that a sustained response is equated with a very favorable prognosis and that it may be equivalent to a cure. There may be other, more subtle benefits of treatment, such as slowing the progression of liver scarring (fibrosis), in patients who do not achieve a sustained response. Thus, slowing HCV replication is advantageous through use of the inventive compositions and methods is of benefit to the subject regardless of whether a sustained response is obtained.

RNAi and siRNA

RNA interference (RNAi) is the process of sequence-specific, post-transcriptional gene silencing or transcriptional gene silencing in animals and plants, initiated by double-stranded RNA (dsRNA) that is homologous in sequence to the silenced gene. The length of a contiguous dsRNA sequence capable of association with and activation of the RNA-induced silencing complex, or RISC, is generally considered to be 19-27 base pairs.

siRNA can be 19, 20, 21, 22, 23, 24 or 25 contiguous nucleotides (nts) in length in a double stranded conformation. There is some evidence that shorter siRNAs have less toxicity; accordingly, each successively shorter length described above is more preferred than the longer one to its right. The length of contiguous nucleotides described does not include any overhang that may be present—thus a siRNA having a sense and a complementary antisense sequence of 19 nucleotides may, in some embodiments, have a single stranded TT overhang at the 3′ end of each sequence. For clarity, therefore, a siRNA will typically have a double stranded center 19-25 nts in length, with 2-nt 3′ overhangs. Exemplar siRNAs of the inventon are set forth in Table 1, within.

As noted, most siRNA are designed with an unpaired, 2 nt overhang on the 3′ end of each strand, or sequence. There have been reports, however, that asymmetric siRNA with one 2 nt overhang and one blunt end are potent RNAi triggers, and that longer, blunt-ended siRNA are more potent than shorter siRNA with 2-nt overhangs. In some embodiments, the siRNAs used in the methods of the invention are asymmetric siRNA. In some embodiments, the siRNAs used in the methods of the invention are asymmetric siRNA. In some embodiments, the siRNAs used in the methods of the invention are siRNA with two blunt ends. It has also been reported that shorter siRNAs may be less toxic than longer siRNAs. The siRNAs used in the studies reported below showed no toxicity or evident off-target effects in a mammalian animal model. In some embodiments, the siRNAs used in the methods of the invention are 19 nt double stranded siRNA with unpaired 2′nt TT overhangs.

The compositions and methods of the invention contemplate the use of combinations of at least two dsRNA or siRNA that have been shown to be surprisingly effective in inhibiting replication of HCV in vitro and in vivo. In some embodiments, the first dsRNA or siRNA has a sense having a sequence of 19-24 contiguous nts of sequences selected from (a) to (f), below:

(a)
(SEQ ID NO: 29)
5′-GCCUUGUGGUACUGCCUGAUAGGG-3′;
(b)
(SEQ ID NO: 30)
5′-CCCCGGGAGGUCUCGUAGACCGUG-3′;
(c)
(SEQ ID NO: 31)
5′-GAGGUCUCGUAGACCGUGCACCAU-3′;
(d)
(SEQ ID NO: 32)
5′-ACCGUGCACCAUGAGCACAAAUCC-3′;
(e)
(SEQ ID NO: 33)
5′-AACCUCAAAGAAAAACCAAAAGAA-3′;

while the sense sequence of the second dsRNA or siRNA also has a sequence selected from SEQ ID NOs:29-33. Since the two dsRNA or siRNA are intended to be different, however, the sense sequence of the second dsRNA or siRNA should not be selected from a sequence identified by the same letter (a-f) as identifies the sense sequence of the first dsRNA or siRNA. In some embodiments, the sense sequence and the antisense sequence of the first siRNA and of the second siRNA, respectively, have a two nucleotide overhang on the 3′ ends of one or preferably of both strands. Preferably, the nucleotides of the two nucleotide overhangs are Ts. In some preferred embodiments, the sense sequence, with overhang, of the first siRNA consists of a sequence selected from the group consisting of SEQ ID NO:11, SEQ ID NO:15, SEQ ID NO:17, SEQ ID NO:19, and SEQ ID NO:23, provided that if the sense sequence selected is SEQ ID NO:17, the sense sequence selected for the sense sequence of the second siRNA is not SEQ ID NO:19. In some preferred embodiments, the siRNAs are selected from the group consisting of the following, provided that if one siRNA is si321, the second is not si333.

si279:
(SEQ ID NO: 11)
5′-CUUGUGGUACUGCCUGAUATT-3′
(SEQ ID NO: 12)
3′-TTGAACACCAUGACGGACUAU-5′;
si315:
(SEQ ID NO: 15)
5′-CCGGGAGGUCUCGUAGACCTT-3′
(SEQ ID NO: 16)
3′-TTGGCCCUCCAGAGCAUCUGG-5′;
si321:
(SEQ ID NO: 17)
5′-GGUCUCGUAGACCGUGCACTT-3′
(SEQ ID NO: 18)
3′-TTCCAGAGCAUCUGGCACGUG-5′;
si333:
(SEQ ID NO: 19)
5′-CGUGCACCAUGAGCACAAATT-3′
(SEQ ID NO: 20)
3′-TTGCACGUGGUACUCGUGUUU-5′,
and
si359:
(SEQ ID NO: 23)
5′-CCUCAAAGAAAAACCAAAATT-3′
(SEQ ID NO: 24)
3′-TTGGAGUUUCUUUUUGGUUUU-5′.

### Measuring Inhibition of Replication of Hepatitis C Virus

Several methods are used in the art for measuring the inhibiting replication of HCV. Inhibition of replication of hepatitis C virus (HCV) is typically measured by the number of viruses or of “viral equivalents,” per milliliter of blood, as determined by a quantitative HCV RNA real time PCR test or the more sensitive (and preferred, for this purpose) qualitative HCV RNA test. Quantitative and qualitative tests are discussed in, e.g., Lee et al., J Clin. Microbiol. 38(11):4171-4179 (2000), Bonetti et al., J Viral Hepat. 4(2):107-112 (1997); Zeuzem et al., Hepatology 27(4):1149-1156 (1998); Pawlotsky et al., Hepatology 27(6):1700-1702 (1998); Morandi et al., J Virol Methods 140(1-2):148-154 (2007); and, Halfon et al., J Med Virol. 78(2):208-215 (2006).

### Nanosomes

The studies underlying the present invention use nanoparticles such as nanosomes to deliver siRNA to cells or to organisms. Nanosomes are well known in the art and are described in, for example, Castor, Current Drug Deliv., 2(4):329-340 (2005), as “nanometer-sized vesicles of phospholipid bilayers.” The term is used here regardless of whether the lipids of the nanoparticles form a bilayer or an internal compartment. For purposes of the compositions and methods of the invention it is important only that the nanoparticles can serve as carriers for nucleic acids and, in particular, siRNAs, and are small enough on average to permit the nucleic acids to enter the liver fenestrae or otherwise to enter liver cells. Studies underlying the present invention revealed that nanosomes with an average size of 150 nm or less were more effective in delivering agents to liver cells. Thus, the invention provides compositions and methods useful in delivering siRNA targeting the HCV 5′UTR to the liver. More generally, the inventive compositions and methods can be used to deliver nucleic acid agents other than siRNA (such as expression plasmids for particular genes) to the liver.

The nanosomes used in the compositions and methods of the invention are composed of non-toxic cationic and neutral lipids. Since siRNA or nucleic acid agents are anionic, the ionic interactions between the cationic lipids and the nucleic acids help keep the siRNA associated with the nanosome and may further facilitate the nucleic acid in crossing the cell membrane into target cells. While the nucleic acids may be sequestered within the nanosomes, some may be complexed to the nanosome by ionic interaction.

Any cationic lipid can be used that is non-toxic and does not interfere with delivery of the siRNA or nucleic acid agent can be used. Preferred cationic lipids are 3β-[N-(N′,N′-dimethylaminoethane)-carbamoyl]cholesterol hydrochloride (DC-cholesterol); 1,2-dioleoyl-3-trimethylammonium-propane (DOTAP); 1,2-dioleoyl-3-dimethylammonium-propane (DODAP); dimethyldioctadecylammonium (DDAB); 1,2-dilauroyl-sn-glycero-3-ethylphosphocholine (Ethyl-PC), or 1,2-di-O-octadecenyl-3-trimethylammonium propane (DOTMA). DOTAP is the most common cationic lipid used for non-viral gene delivery.

Further, the nanosomes comprise one or more non-toxic neutral lipids. Preferably, the neutral lipid is cholesterol. Inclusion of cholesterol facilitates formation of stable lipid vesicles. The studies reported in the Examples used DOTAP:cholesterol at a 1:1 ratio. Preferred ratios of cationic lipid to cholesterol are 2:1 to 1:3. Lower ratios of cationic lipid to neutral lipid are preferred, as there is less chance of toxicity in in vivo administration. For DOTAP:cholesterol nanosome, a 1:1 ratio is particularly preferred. DOTAP-cholesterol nanosomes are particularly preferred in the compositions and methods of the invention.

The nanosomes may be formed by conventional techniques. Typically, the lipids are mixed in chloroform and the chloroform is evaporated to provide a lipid film. Deionized water is added to form the nanosomes. As noted below, other cationic polymers may also be present, and the water may further contain a sugar if the nanosomes are going to be lyophilized or spray dried before use.

DOTAP-cholesterol liposomes were used in a clinical trial which commenced in 2003 and is now completed. That trial introduced an expression plasmid for a tumor suppressor gene, “fusl” (also called “TUSC2”) into patients with non-small cell lung cancer. Information regarding the clinical trial, number NCT00059605, is set forth on the government's clinical trial website, which can be located by entering the clinical trial number above into a web browser.

In some embodiments, the nanosomes may be prepared, mixed with the nucleic acid agent of choice, and administered to the subject. In other embodiments, it may be desirable to prepare the nucleic acid-nanosome compositions and then store or transport them. In these embodiments, the nucleic acid-nanosome compositions may be prepared for storage by conventional techniques such as, for example, lyophilization or spray drying. Nucleic acid-nanosome compositions that are to be lyophilized or spray dried are typically added to a sugar solution to preserve the structure and integrity of the nanosome. Suitable sugars include trehalose, sucrose, lactose and mannitol, with trehalose being particularly preferred. Typically, the lipid:sugar ratio for rehydration is 1:0.5-10. The nucleic acid-nanosome compositions are then lyophilized or spray dried. The lyophilized or spray dried compositions are then rehydrated before administration.

### Cationic Polymers

In some embodiments, the nucleic acids, such as siRNAs, are condensed prior to being complexed with or encapsulated withing the nanosomes. In preferred embodiments, the nucleic acids are condensed with a non-toxic cationic polymer such as protamine sulfate, chitosan, polyethylene imine (“PEI”, 0.01-1%), or cetyl trimethylammonium bromide (0.01-1%). Chitosan (deacetylated chitin biopolymer) is commercially available from, for example, Sigma-Aldrich (St. Louis, Mo.) at molecular weights (mw) designated as being low, medium or high. The siRNA:chitosan ratios for use in the compositions and methods are, by weight, for low mw chitosan, siRNA:chitosan of 1:0.5-3, for medium mw chitosan, siRNA:chitosan of 1:0.2-2, and, for high mw chitosan, siRNA:chitosan of 1:0.1-1. PEI is less preferred for administration to subjects. Protamine sulfate is considered safe at the concentrations used in the compositions and methods of the invention and is a preferred cationic polymer.

### Sonication

Conventional methods of preparing nanosomes include, for small batches, sonication and, for large scale production, high pressure homogenization. The nanosomes are conventionally prepared for administration by, for example, drawing them through syringes to break up any aggregations that have accumulated.

During the course of some of the studies reported herein, it was noted that the liver fenestrae were reported to have a size only a little larger than 150 nm Entry through the fenestrae is one key to successful transfection of liver cells. In studies underlying the present invention, we examined the size of the nanosomes prepared by high pressure homogenization and found a wide variation. While some of the nanosomes were below the 150 nm threshold for entering liver cells through the fenestrae, at least 30% and more probably 50% were too large to enter liver cells by this route. This meant that a substantial portion of any nucleic acid-nanosome preparations administered to subjects would consist of nanosomes too large to readily enter liver cells, requiring a larger dose of nucleic acid-nanosome to achieve any desired therapeutic effect.

Sonicating the nucleic acid-nanosomes in a bath sonicator prior to administration notably increased the percentage of nanosomes below the 150 nm threshold for the liver fenestrae. As shown in the studies reported below, sonication did not cause the nucleic acids (in this case, siRNA) to dissociate from the nanosomes or disrupt the nucleic acids and prevent them from inhibiting HCV replication. As shown in the studies reported below, sonicating the compositions for as little as 30 seconds markedly reduced the average size of the nanosomes. The reduction in the average size of the nanosomes plateaued after a few minutes. While sonication of more than 5 minutes can be performed if desired, it is not necessary as the studies showed sonication of from 30 seconds to 5 minutes gave satisfactory results in reducing average nanosome size.

The nucleic acid-nanosome compositions can be sonicated in a bath sonicator immediately before administration to a subject, but cannot be sonicated so long before administration that the sonicated nucleic acid-nanosomes have a chance to reaggregate before they are administered. As reaggregation can occur if the nucleic acid-nanosomes have more than approximately 1.5 hours before administration, the nucleic acid-nanosomes should be administered within approximately (with approximately here meaning about 5 minutes on either side)1.5 hours of sonication, with each shorter 5 minute period between sonication and administration (i.e., 1 hour 20 minutes, 1 hour 15 minutes) being more preferable down to immediately before administration. Sonication between 5-45 minutes before administration is a preferred time as the window does not place an undue time pressure on the practitioner, but keeps the time after sonication short enough so that reaggregation will not have occurred before the compositions are administered.

### Administration

In some embodiments, the invention contemplates introduction of the compositions of the invention to liver cells. In other embodiments, the invention contemplates the use of sonication of nucleic acid-nanosome constructs to introduce nucleic acids to liver cells of interest.

Considerable effort over the past decade has gone into methods of administering nucleic acids to liver cells, either for expression of a protein encoded by the exogenous nucleic acid or to silence protein expression in the cell. Kamimura and Liu, AAPS J 10(4):589-595 (2008), for example, review some of the methods employed to deliver nucleic acids to the liver, including direct injection into the liver, intraportal or intrahepatic vein, intrahepatic artery, intrabile duct, or systemic administration. Any of these methods can be used to administer to liver cells compositions of the invention or sonicated nanosomes bearing nucleic acids. In some embodiments, intravenous administration is a preferred method for administration of the compositions of the invention or for administration of sonicated nanosomes. The nucleic acid are generally administered in a dose of 0.01 mg/kg to 0.09 mg/kg.

### EXAMPLES

### Example 1

This Example sets forth the materials and methods used in the studies reported and discussed in Examples 2 and 3.

Cell culture of HCV and viruses. The Huh-7.5 cell line was obtained from the laboratory of Charlie Rice (The Rockefeller University, New York). Interferon resistant replicon cell lines were generated in our laboratory by prolonged treatment of low inducer HCV replicon cell lines with IFN-a as described previously. Hazari et al., Virol J 7:36 (2010) (“Hazari 2010”). A cured Huh-7 cell line (R-24/1) with defective a Jak-Stat pathway was prepared from an IFN-α resistant replicon cell line (R-Con-24/1) after repeated treatment with cyclosporine-A (1 μg/ml). The JFH1 full-length and sub-genomic cDNA clone of the HCV 2a strain was obtained from Dr. Wakita (National Institute of Infectious Diseases, Tokyo, Japan) (Wakita et al., Nat Med 11:791-796 (2005)). Chimeric clones between JFH1 (both full-length and sub-genomic) and enhanced green fluorescent protein (EGFP) were constructed in our laboratory. R4-GFP is an IFN-a resistant HCV-GFP chimera replicon cell line that was developed using R-24/1 cured Huh-7 cell line. This cell line stably replicates GFP-tagged sub-genomic HCV RNA of HCV2a. Replication of HCV in R4-GFP cell line is resistant to IFN-α due to defective Jak-Stat signaling. Huh-7 and Huh-7.5 cells were cultured in DMEM with 10% FBS as described by Hazari 2010, supra. A Renilla luciferase reporter based full-length HCV clone (JFH-ΔV3-Rluc) was obtained from the laboratory of H. Hagedorn (University of Utah School of Medicine, UT) (Liu et al., Antiviral Res 89:54-63 (2011)). In this clone, the V3 region of NS5A is replaced with the Renilla reniformis luciferase (Rluc) gene, resulting in an infectious virus that stably expresses an NS5A-Rluc fusion protein. Cells infected with this clone provide a robust luciferase signal that accurately reflects the production of infectious virus. Cell culture derived infectious HCV stocks were prepared from the supernatants of infected Huh-7.5 cells as described by Hazari 2010, supra.

siRNA Targets. Thirteen siRNAs were selected to target the highly conserved 5′ UTR of the JFH1 clone (GenBank accession No. AB114136). The nucleotide sequence of each siRNA is shown in Table 1, below, and in FIG. 1A, and the targeted location in the 5′ UTR of the HCV genome is shown in FIG. 1A. Table 1 also shows the sequence of a control siRNA, “siIRR,” targeted to the Epstein-Barr virus nuclear antigen and not expected to affect HCV replication. Synthetic siRNAs were purchased in gram quantities from Invitrogen Corp., Carlsbad, CA. The length of each siRNA is 21 nucleotides with 19 matching sequence and a TT overhang. Cy3-labeled siRNA targeted to GAPDH mRNA was purchased from Ambion Inc., Austin, Tex.

Preparation of siRNA-nanosome: Briefly, nanosomes were prepared using 1:1 molar ratio of cholesterol (Avanti Polar-lipids Inc., Birmingham, Ala.) and 1,2 dioleoyl-3-trymethylammonium-propane (DOTAP). The final concentration of lipid in the nanosome preparation was 20 nM. DOTAP (50 mg, Avanti Polar-lipids Inc.) and cholesterol (26.7 mg) were dissolved in 20 ml of HPLC grade chloroform (Sigma Chemical Co. St. Louis, Mo.) in a round bottom flask and vacuum dried under nitrogen gas. On the following day, the resulting films of the lipids were hydrated by the addition of 10 ml of distilled water. The lipid dispersion was homogenized by using an EmulsiFlex®-B3 high-pressure homogenizer (Avestin Inc., Ottawa, Canada) at 20,000 PSI for 5 cycles. The siRNAs were encapsulated in the nanosomes by using protamine sulfate (Sigma Chemical Co.). First, siRNAs were mixed with protamine sulfate in distilled water by incubating at room temperature for 40 minutes. Second, the siRNA-protamine sulfate complex was added drop-wise to the nanosome and the solution was mixed thoroughly. Finally, the siRNA-nanosome complex was resuspended in 0.3 M trehalose (Sigma Chemical Co.) and used immediately.

Cell viability assay. The antiproliferative activity of siRNA-nanosome complexes was measured by 3-(4,5-dimethylthiazol-2-yl-2,5-diphenyltetrazolium bromide (MTT) assay (Sigma-Aldrich, Saint Louis, Mo.). In brief, cells were seeded in 24 well plates at a density of 2×10⁴ cells per well, after 24 h siRNA-nanosome complexes were added to each well. The siRNA concentration was varied from 25 pmole to 200 pmole. The MTT assay was performed in triplicate 48 hours post-transfection.

IFN-β promoter activity. Activation of the endogenous IFN-promoter in siRNA treated cultured cells was assessed by measuring the ISRE-luciferase activity (Hazari et al., supra). Equal numbers (4×10⁴ cells/well) of Huh-7.5 cells were seeded in a 24-well plate, then the next day 0.5 μg of pISRE-Firefly luciferase reporter plasmid was transfected by FuGENE®-6 (Roche Diagnostics, Indianapolis, Ind.). Increasing amounts of siRNA-nanosome complexes were added 2 hours post-transfection. After 24 hours, luciferase activity was measured. IFN-α treatment was used as a positive control.

siRNA-nanosome treatment in the replicon model. R4-GFP cells (2×10⁵ cells/well) were treated with nanosomes containing 13 HCV specific and one control siRNA (siIRR, targeted to Epstein-Barr virus nuclear antigen). The antiviral effect of each siRNA was first examined by detection of GFP fluorescence, which was subsequently quantified by flow cytometry. The long-term sustained antiviral activity of the siRNA-nanosome treatment was measured by the appearance of G-418 resistant colonies and a reduction of HCV RNA levels by RT-qPCR. Complete inhibition of HCV replication was assessed by nested RT-PCR followed by Southern blotting. GFP expression was monitored under the Olympus 1X70 microscope (Olympus America Inc., Center Valley, Pa.) using the ×20 objective and a DP-71 digital camera. The exposure time was 200 ms. Adobe® Photoshop® 7.0 imaging software was used for the acquisition of microscopic images.

siRNA-nanosome treatment in the infection model: Inhibition of HCV replication in infected cell cultures after siRNA-nanosome treatment was determined by performing a multi-cycle infectivity assay. To screen the antiviral efficacy of the 13 siRNAs, 100 pmole of an individual siRNA were transfected and, after 72 hours, the luciferase activity was measured. Complete clearance or the development of an escape mutant virus was assessed by infectivity assay as described herein.

Escape mutant analyses: Replicon cell clones that escaped the siRNA-nanosome treatment developed G-418 resistance. The G-418 resistant colonies were picked and expanded into stable cell lines. Total RNA was isolated from the G418 resistant cells the 5′UTR sequence was PCR amplified, cloned and sequenced. Escape mutants were identified by sequence analysis using Bioedit version 7.0.4.1 (Hall, Nucleic Acids Symp Ser 41:95-98 (1999).

siRNA-nanosome treatment in mice. The success of intracellular delivery of the siRNA-nanosome formulation was examined using a HCC tumor xenograft mouse model for HCV developed in our laboratory. Hazari et al., World J Gastroenterol 17: 300-312 (2011) (“Hazari 2011”). Female NOD/SCID mice of 6-8 weeks old were used for tumor xenografts using the mouse adapted HCV sub-genomic replicon cell line developed in our laboratory. Mice were obtained from Charles River Laboratory and maintained at the Department of Comparative Medicine, Tulane University Health Sciences Center. All animal experiments were carried out after receiving approval from the Institutional Animal Care and Use Committee (IACUC), Tulane University Health Sciences Center.

Development of a murine subcutaneous tumor model. In vivo testing of the siRNA-nanosome was performed using 4 groups of mice with each group consisting of 5 mice. One million mouse-adapted GFP replicon cells were implanted subcutaneously into the right and left flank of mice which were monitored for the development of tumors. When the tumors reached 10 mm in size, they were injected with a siRNA-nanosome at 5 mg/kg body weight. The first group of animals was used to examine the biodistribution of Cy3-labeled siRNA-nanosome complex targeted to GAPDH mRNA in the tumor. The peritumoral injection of siRNA was performed using a 27-gauge needle. After 6 hrs, the mice were euthanized and frozen sections of the subcutaneous tumors were prepared. Frozen tissue section (10-micron) of sub-cutaneous tumors were prepared, washed in PBS, fixed in 4%-parformaldehyde for 30 minutes and then counter stained with Hoechst dye (H33342, Calbiochem, Darmstadt, Germany) for 15 minutes at 37° C. Tissue sections were then examined at 340 nm for blue nuclear staining. The same areas of the tissue section were examined at 484 nm using a fluorescence microscope (Olympus) for expression of green fluorescence. For each area, two sets of pictures were taken. The final image was generated by superimposing the blue nuclear fluorescence with the cytoplamsic green fluorescence of GFP using Abode® Photoshop® software (V 7.0). The second group was used as an untreated control and the third group received a peritumoral injection of control siRNA-nanosomes. The fourth group of mice received peritumoral injections of HCV-specific si321 and si359 encapsulated with nanosomes. After six injections, the antiviral effect against HCV was measured by a number of assays: GFP expression, colony formation, quantification of viral RNA by RPA and real-time RT-qPCR.

Development of a murine liver tumor model. Development of a murine liver HCC tumor xenograft has been published previously by our laboratory. Hazari 2011, supra. A total of 15 mice harboring liver tumor were divided equally into 3 groups. One group remained untreated, another 2 groups were treated with 5 mg/Kg body wt nanosome encapsulated control siRNA or nanosome-encapsulated si321 and si359 in combination (2.5 mg/Kg body wt of each). The treatment began 28 days after tumor cell injection. A total of 100-μl of siRNA-nanosome was injected through the tail vain for 6 consecutive days. After 34 days, the mice were sacrificed and liver tissues were processed immediately for analysis. After dividing each liver lobe into three parts, colony assay were performed on a part from each lobe by culturing the recovered cells in 1 mg/ml G-418. Another portion of liver tissue was used for RNA isolation in order to measure the HCV RNA levels by RPA and RT-qPCR. The remaining liver tissue was used to prepare frozen sections which were stained with hematoxylin and eosin.

Toxicity of siRNA-nanosome administration in BALB/c mice. A total of 35 female BALB/c mice (6-weeks old) were used to assess the toxicity of siRNA-nanosome administration. The animals formed 7 groups (n=5/group). One group was used as an untreated control to establish base line values. Groups of mice were injected by tail vain with 100 μl of siRNA-nanosome (EBNA1) at a dose of 5 mg/kg of body weight. Another group of mice was injected with 100 μl of saline as control. The mice were sacrificed at 4 hours and 24 hours to assess the acute toxicity. After 4 h or 24 h of post injection, blood and organs were collected from the treatment and control groups (n=5/group). A group of 5 mice was injected every other day with either saline or siRNA-nanosome for a total of 3 injections and sacrificed on day 8 to assess the toxicity due to this repeated treatment. Blood and other tissues (heart, lung, liver, spleen and kidney) were collected. Control animals injected with saline were treated similarly. Serum was obtained by centrifugation of the whole blood at 3000 rpm for 15 min at 4° C. Hematoxylin and eosin (H&E) stained tissue sections were examined by two pathologists for toxicity. Serum enzyme alanine aminotransferase (ALT), and aspartate aminotransferase (AST) levels were measured in the Pathology clinical laboratory, Tulane Medical School. Body weights were obtained to assess systemic toxicity in mice.

Statistical analysis. The student's t-test (paired, two-sample unequal variance) was used to compare the inhibition efficacy of HCV specific siRNAs between treated and untreated mice in both in vitro and in vivo studies. The differences were considered statistically significant when P<0.05 (two-tailed distribution).

Quantification of HCV RNA level by Real-Time PCR (RT-qPCR): Assay was performed by using a modified protocol published in Hazari 2010, supra. Briefly, 2 μg of cellular RNA or total RNA from 1 ml culture supernatant was used to amplify the 5′-UTR region of HCV genome using sense primer 5′-TCTTCACGCAGAAAGCGTCTA-3′(60-80; HCV/S) (SEQ ID NO:36) and anti-sense primer 5′-CGGTTCCGCAGACCACTATG-3′ (157-138; HCV/AS) (SEQ ID NO:37). The probe (5′/56-FAM/TGAGTG TCG/ZEN/TGCAGCCTCCAGGA/3IBκFQ/-3′) (SEQ ID NO:38) labeled at the 5′ end with a FAM (6-carboxyfluorescein) fluorophore reporter molecule and ZEN®-Iowa Black® FQ (IBFQ) double quenchers (Integrated DNA Technologies, Coralville, Iowa) was used to reduce the background and increase signal than traditional dye-quencher combinations. RT-qPCR assay was performed in 20 μl containing 10 μl of iQ® supermix (Bio-Rad Laboratories Inc., Hercules, Calif.), 0.25 μM of each primers and probe and 4 μl of cDNA product obtained from the RT reaction. The amplification was carried out using the standard program that includes: the first cycle at 48° C. for 30 minutes, 95° C. for 10 minutes, then additional 45 cycles each cycle consists of a denaturation step at 95° C. for 15 seconds, annealing and extension step at 60° C. for 1 minute. HCV cDNA standards were used starting at 10⁹ copies of virus and decreasing in 10-fold serial dilutions. Amplification, data acquisition, and analysis were performed on a CFX96™ Real-Time instrument using CFX Manager® software (Bio-Rad Laboratories Inc.).

siRNA-nanosome treatment of HCV infected cells: Huh-7.5 cells (1.2×10⁵ cells) were infected with 1 ml of culture supernatant containing either JFH1-GFP or JFH1-ΔV3-Rluc virus (MOI 0.1) for 24 hours. On the following day, the infected culture was washed with PBS and then incubated with freshly prepared media. To screen the antiviral efficacy of 13 designed siRNAs, 100 pmole of individual siRNA was transfected and after 72 hours measured the luciferase activity. The complete clearance or the development of an escape mutant virus was assessed by infectivity assay. Cells received two consecutive treatments within 5 days were then cultured up to 30 days. The effect of the siRNA treatment on the replication of the full-length virus in the culture was measured by NS5A-Rluc activity. Total protein concentration was measured by the Bradford method and the luciferase activity was expressed in per micro-gram of total protein. Western blots and immunostaining for HCV-core were performed using monoclonal antibody (Thermo Fisher Scientific, Logan, Utah). The HCV-RNA level in the infected cells was measured by RT-qPCR.

Escape Mutant Analysis: Total cellular RNA was isolated from resistant as well as untreated replicon cell lines by the GITC method. The HCV RNA sequences in the sub-genomic RNA clone (Accession No AB114136), starting from nucleotide 10 to nucleotide 496 that include the siRNA targets, were amplified by reverse transcription hemi-nested PCR reaction using oligonucleotides 5′-UTR/10S: 5′-AATAGGGGCGACAC TCCGCCA-3′ (SEQ ID NO:39), G-418/99AS: 5′-ATCAGAGCAGCCGATTGTCTG-3′ (SEQ ID NO:40) and primer 5′-UTR/43S: 5′-CTGTGAGGAACTACTGTC-3′ (SEQ ID NO:41). The amplified DNA fragment was purified from an agarose gel and cloned into the plasmid pCR2.1 using the TA cloning kit according to the manufacturer's recommendation (Invitrogen, Carlsbad, Calif.). Three plasmid clones from each cell line were sequenced using the M13 forward and reverse primers then sequences were edited, aligned and analyzed using Bioedit version 7.0.4.1. Presence of the escape mutant clone was determined by comparing the DNA sequence of the mutant clone with the wild type replicon. Appearance of escape mutant virus in the infected cell culture was determined by muticycle infectivity assay using Renilla luciferase.

Immunostaining for HCV core: Infected Huh-7.5 cells with or without siRNA treatment were mounted onto a glass slide via the cytospin method. The cells were washed twice with 10 mM PBS pH 7.4 (Sigma-Aldrich) for 5 minutes. The cells were fixed in chilled acetone for 15 minutes and then permeabilized by treatment with Reveal Decloaker, RTU (RV100, Biocare Medical LLC, Concord, Calif.) for 25 minutes at boiling point. Slides were then cooled down to room temperature for 25 minutes. Blocking was performed utilizing Background Sniper (Biocare Medical, BS966) for 10 minute at room temperature. The cells were incubated with monoclonal anti-core antibody (Thermo Scientific, Pierce hepatitis C virus core antigen specific mouse monoclonal antibody, Mal-080) at 1:200 diluted with DaVinci Green Diluent (Biocare Medical, PD900) for 1 hour at room temperature. Following the primary antibody incubation, the cells were washed 3 times in Tris Buffered Saline (pH 8.0), and incubated with MACH 4™ mouse probe (Biocare Medical, UP534) for 10 minutes. After mouse probe treatment, the cells were incubated with MACH 4™ HRP Polymer (Biocare Medical, MRH534) for 30 minutes, and washed with TBS 3 times. Next, the cells were treated with diaminobenzidine (DAB) chromogen (Dako Cytomation, Carpinteria, Calif.) for 5 minutes. The slides were counterstained with hematoxylin for 30 seconds and Tacha's Bluing Solution (Biocare Medical, HTBLU) for 30 seconds, dehydrated, mounted and observed by light microscopy.

### Example 2

This Example discusses the results of studies using the materials and methods discussed above.

Design of multiple siRNA targets and formulation of siRNA-nanosome: Thirteen different siRNA duplexes targeting the stem-loop domains II-IV of HCV 5′UTR sequences of the JFH1 clone were chemically synthesized. The siRNA sense and antisense sequences are listed in Table 1. The full target sequences, with respect to the predicted secondary structure of the 5′UTR of the HCV genome, are shown in FIG. 1a. Endogenous cellular microRNA-122 also directly binds to two locations in the 5′UTR of HCV and positively regulates internal ribosome entry site (IRES)-mediated translation. The two miR-122 binding sites located in the 5′UTR of HCV are distinct from the siRNA targets used in our study. Lipid nanoparticles (nanosomes) were prepared using a mixture of cholesterol and DOTAP. Individual siRNA molecules were encapsulated within nanosomes following condensation with protamine sulfate. The siRNA-nanosome formulations were sonicated to reduce the particle size to 100 nm and zeta potential of 10-15 mV. Our results show that sonication of siRNA-nanosome formulations result in higher liver deposition and gene silencing properties without changing the zeta potential of lipid nanoparticles or siRNA encapsulation. The efficiency of siRNA delivery and intracellular stability were determined by fluorescence microscopy and flow cytometry using Cy3-siRNA targeted to GAPDH mRNA. Nanosomal delivery of siRNA to cells in culture was 100% efficient, and siRNA was stable intracellularly for more than 7 days. At 200-pmole concentrations of siRNA-nanosome, 88.4% of cells were viable, as determined by MTT assay. The activation of the interferon response and endogenous IFN-β production due to intracellular delivery of siRNA were examined using interferon-sensitive responsive element (ISRE)-firefly luciferase reporter plasmid in an interferon-sensitive cell line (Huh-7.5). The results shown in FIG. 1f exclude the possibility of activation of the endogenous interferon system due to siRNA-nanosome treatment.

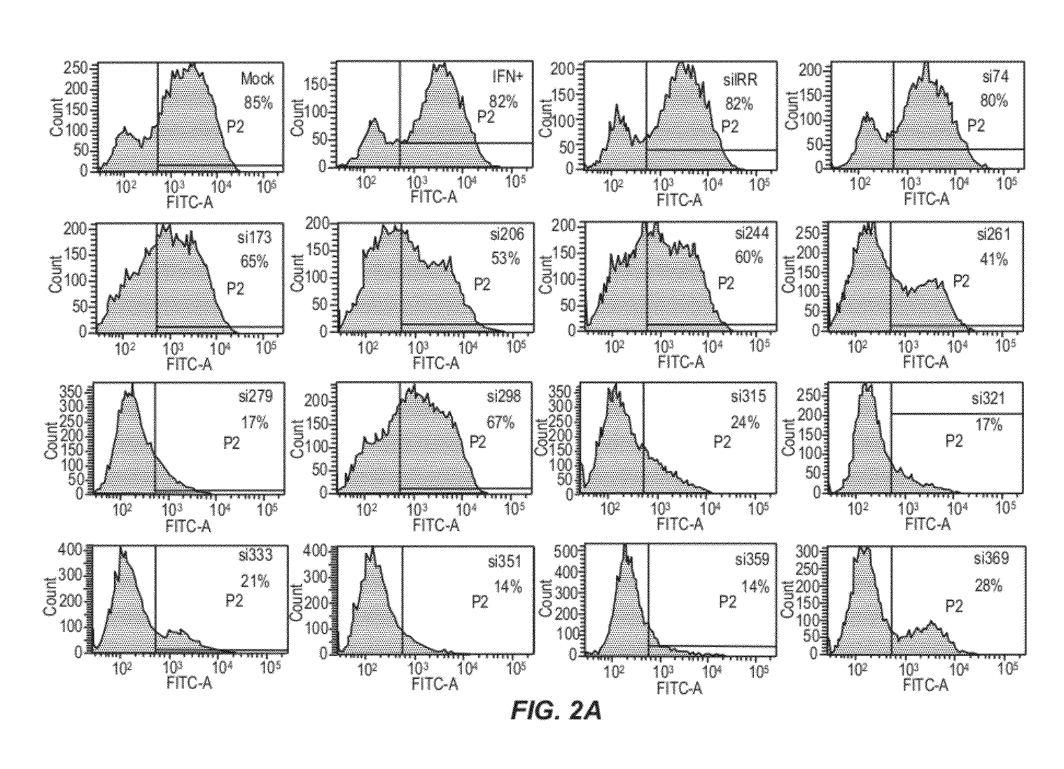

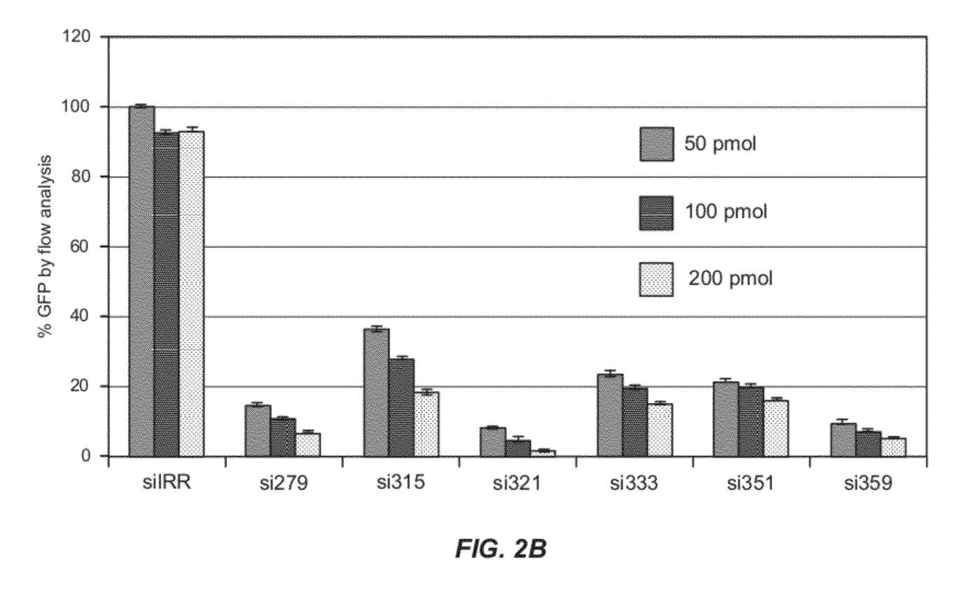

Antiviral effect of siRNA-nanosome using GFP-replicon cell line: The antiviral effect of 13 different siRNAs was determined using a green fluorescent protein (GFP) reporter-based HCV sub-genomic replicon cell line (R4-GFP). In Hazari 2010, supra, we published that defective Jak-Stat signaling due to expression of truncated IFNAR1 in this cell line makes HCV RNA replication resistant to IFN-α. The replicon cell line was treated with an individual siRNA-nanosome, and inhibition of GFP expression was monitored under a fluorescence microscope. We used highly specific assays in the initial screening steps to identify the best target of the 13 siRNAs in the inhibition of HCV replication. At 100 pmole concentrations, six siRNAs—si279, si315, si321, si333, si351, and si359—effectively inhibited HCV replication. Flow cytometric analysis indicated that more than 80% of HCV-GFP expression was reduced after a single treatment of the aforementioned 6 siRNAs. Among the 13 siRNAs tested, 6 showed strong antiviral effects by fluorescence microscopy and flow cytometry (FIG. 2A). Unrelated control siRNA (siIRR) targeted to either Epstein-Barr virus nuclear antigen 1 (EBNA1) (Yin and Flemington, Virol 82:2895-2903 (2006)) did not inhibit GFP expression, as determined by fluorescence microscopy or flow cytometric analysis. The antiviral effects for the 6 siRNAs were also assessed by flow cytometric analysis after 2 consecutive treatments and found to be concentration-dependent (FIG. 2B). Among the 6 siRNAs that substantially inhibit HCV RNA replication, 3 (si279, si321, and si359) showed a strong antiviral response compared to the other siRNAs (si315, si333, and si351), suggesting that their antiviral efficacy may be related to target accessibility in the stem-loop structure of the HCV 5′UTR.

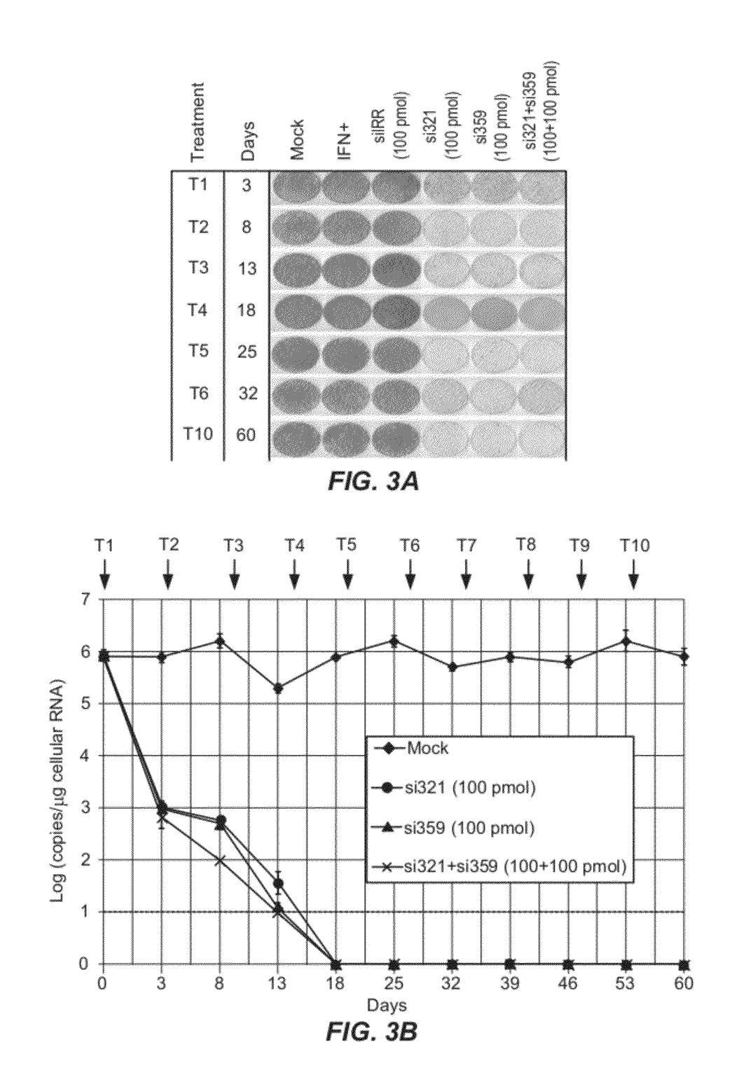

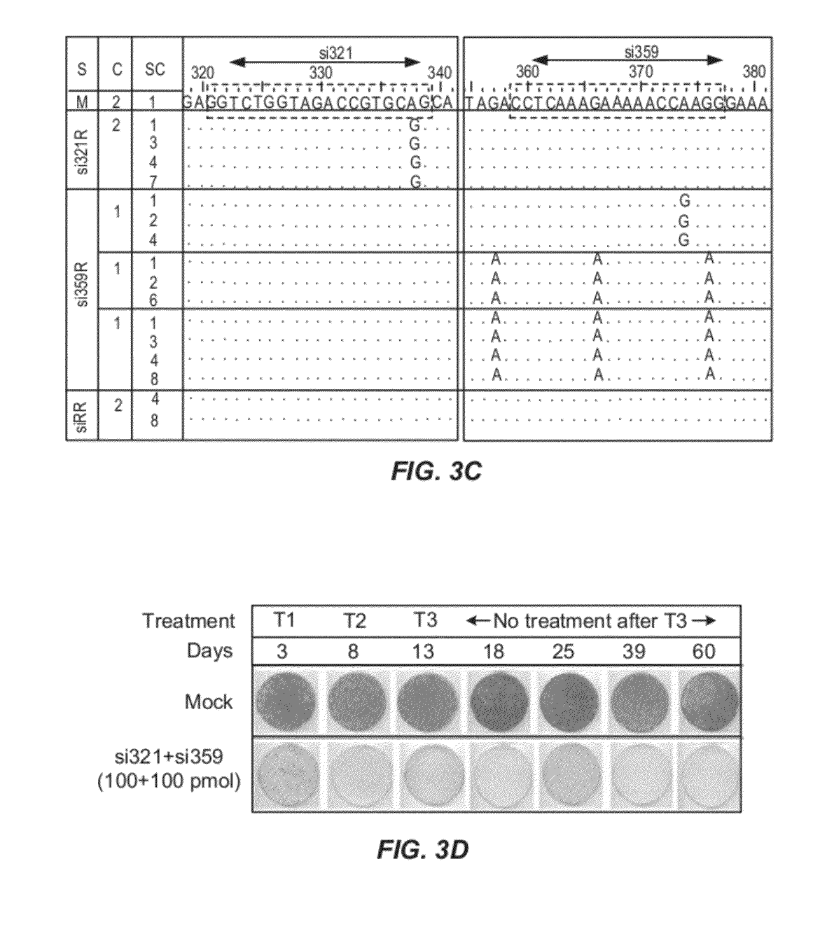

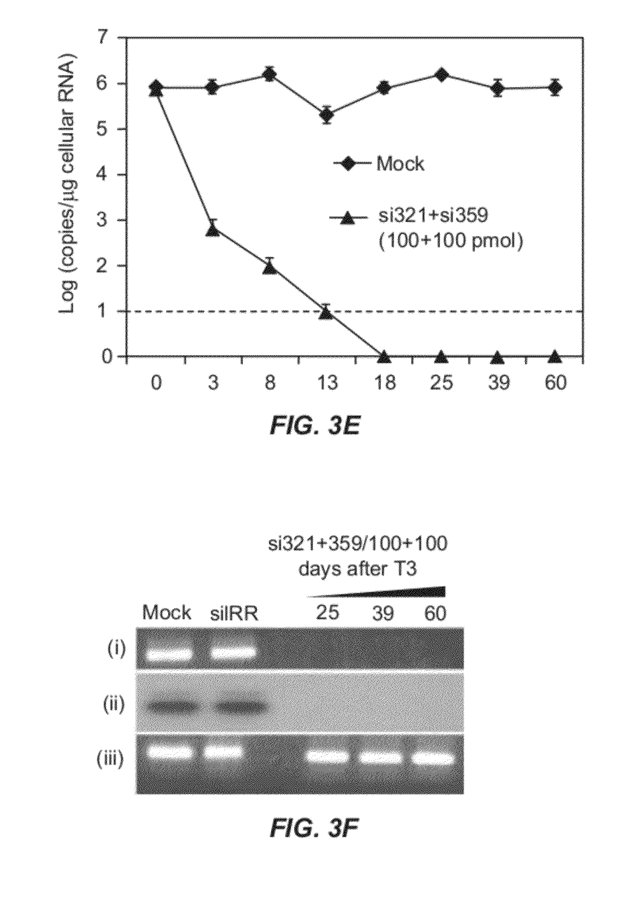

Repeated treatment using 2 siRNAs minimizes escape mutant resulting in rapid inhibition of HCV in the R4-GFP replicon cell line: Inhibition of HCV replication in a R4-GFP cell line by single versus combination siRNA treatments was examined. For this purpose, R4-GFP cells were treated with si321 or si359, alone or in combination. Cells were repeatedly treated with 100 pmole of siRNA-nanosome at 5-day intervals. The antiviral effects of single and combination siRNA treatments on HCV replication in the R4-GFP cell line were determined by colony assay and measuring HCV RNA levels by real-time RT-PCR (RT-qPCR). The replication of HCV in the replicon cell line was assessed by measuring the number of Huh-7 cell colonies that survived the G-418 drug selection. The number of G-418 resistant cell colony is directly proportional to the replication of HCV sub genomic RNA. A smaller number of colonies means a strong antiviral effect of the siRNA treatment, while a larger number of colonies means a lower antiviral effect. The G-418 resistant cell colony assay was used to examine the effect of siRNA treatment on HCV replication. HCV RNA that survived siRNA treatment due to virus escape mechanisms develops G-418 resistant cell colonies. The results of long-term single and combination siRNA treatment on viral replication are shown in FIGS. 3a and b. The combination treatment more effectively inhibited HCV replication within 8 days, as no G-418-resistant cell colonies were found. However, repeated treatment with a single siRNA (si321 or si359) led to the development of G-418-resistant mutant cell clones that could no longer be inhibited by the same siRNA.

To understand the mechanisms of resistance after a single siRNA treatment, a few resistant clones were isolated and stable cell lines were developed. Variations in the siRNA target region were identified by DNA sequence analysis. All four resistant clones (si321R) isolated from si321 treated cells showed A-G substitution in the siRNA target. Three resistant clones (si359R) isolated from si359 treated cells showed two substitutions within the siRNA target and two outside the siRNA target sequence (FIG. 3C). The nucleotide changes were either G-A or A-G transitions. Similar nucleotide changes were not observed in mock- or siIRR-treated cells, suggesting that nucleotide changes within the siRNA target could be the reason for virus escape. The significance of mutations outside the siRNA target is not clear. In HIV, this type of escape mutation pattern outside the siRNA target site has been reported to be due to a change in RNA secondary structures. Von Eije et al., J Virol 82:2895-2903 (2007); Westerhout et al., Nucleic Acids Res 33:796-804 (2005). In our study, visual inspection of the si359 in the HCV 5′UTR does not show such a scenario. Another possibility is that the three G-to-A changes found in the si359 resistant clones are suggestive of an APOBEC-like mutational action reported in HIV-1 studies. Wood et al., PLoS Pathogens 5:e1000414. To prove that the combination siRNA treatment cleared HCV from the replicon cells, the siRNA treatment was terminated after 3 treatments (T3) and cells were studied up to an additional 60 days. The results of the cell colony assay confirmed that no cells survived in the presence of G-418, indicating effective clearance of HCV replication (FIG. 3D). Total RNA was isolated from the cells at 0, 3, 8, 13, 18, 25, 39, and 60 days of siRNA treatment and HCV RNA levels were quantified by RT-qPCR (FIG. 3E) Inhibition of HCV in the siRNA-treated R4-GFP replicon cells was confirmed by RT-nested PCR assay, followed by Southern blot analysis using primers targeted to the HCV 5′UTR (FIG. 3F). The sensitivity of this assay had been determined previously in our laboratory to be 1-10 HCV RNA molecules. We could not detect HCV RNA in the cells after 3 rounds of treatment with si321 and si359, indicating that the culture was free of HCV.

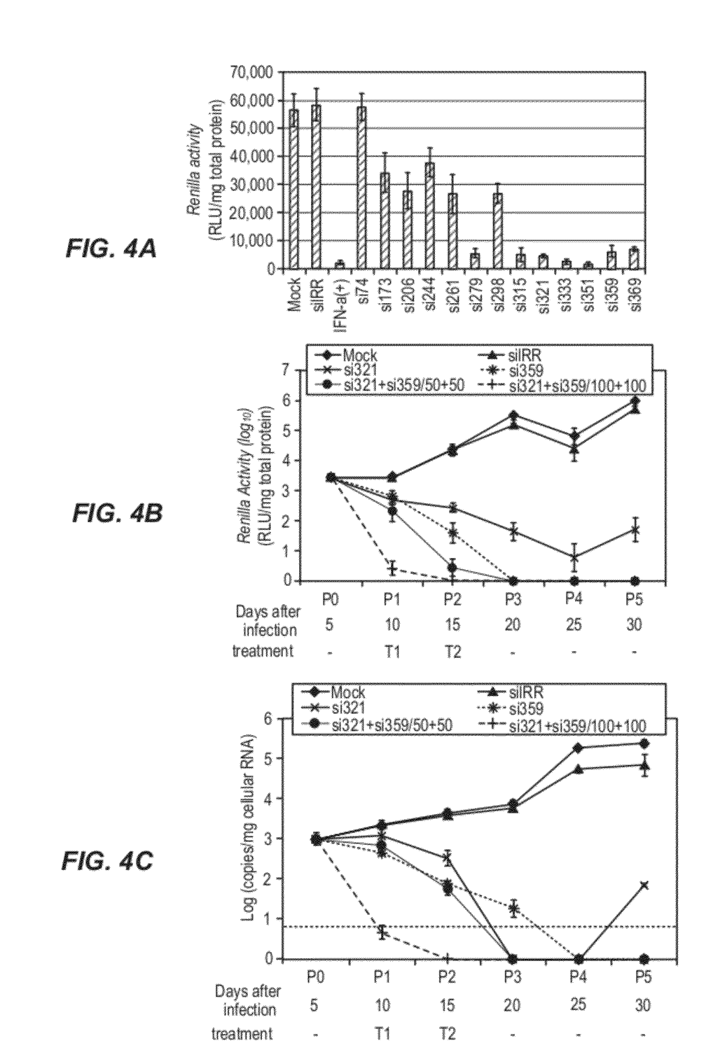

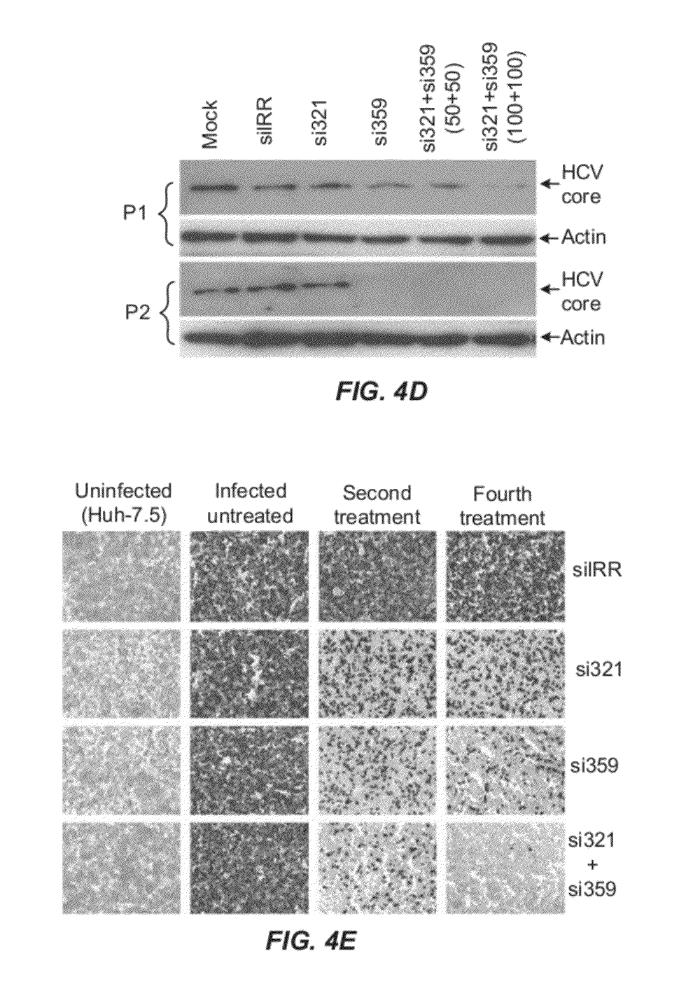

Rapid inhibition of HCV from infected cells by repeated treatment of a combination of 2 siRNAs: The antiviral efficacy of combination siRNA-nanosome treatment was examined using an infectious HCV cell culture system. Liu et al., Antiviral Res 89:54-63 (2011). Cells were infected with either JFH1-GFP or JFH1-ΔV3-Rluc chimera virus at an MOI of 0.1 for 72 hours and then treated with either a single siRNA or a combination of two siRNAs. The siRNA-nanosome complex (100 pmole) was added directly to the infected culture, and HCV replication in the siRNA-treated cells was quantitated by measuring Renilla luciferase expression. Consistent with the results of the replicon cell line, the same 6 siRNAs (si279, si315, si3 21, si333, si351, and si359) showed strong antiviral activity in the infectious cell culture model (FIG. 4A). Additionally, si369 also substantively inhibited HCV replication.

The effects of repeated administration of a single siRNA versus a combination of 2 siRNAs on HCV replication were examined by performing a multi-cycle infectivity assay. Compared to a single siRNA, the combination of 2 siRNAs was highly effective and led to rapid inhibition of HCV in the infected cell culture (FIG. 4B). The antiviral effect appears to be concentration-dependent, because a more substantial inhibition of HCV replication was observed at 100 pmole siRNA than at 50 pmole. The levels of HCV RNA in the combination siRNA-treated group remained below the level of detection threshold after 2 treatments (FIG. 4C). The HCV RNA levels in the infected culture after siRNA treatment was followed for 5 infectivity cycles. Combination treatment with si321 and si359 decreased the total HCV RNA level; the virus became undetectable after the third passage (FIG. 4C). The levels of HCV remained below the detection threshold for over 1 month, whereas HCV levels remained detectable in culture cells treated with a single siRNA. The siRNA-nanosome complex (100 pmole) was added directly to the infected culture. The resulting inhibition of GFP expression that was monitored by fluorescence microscopy indicates the inhibition of HCV replication.

The efficacies of a combination of other siRNAs in treating HCV infection were examined. The results were also confirmed by western blot analysis for HCV core protein (FIG. 4D). These results indicate that inhibition of HCV replication in the infected culture can be achieved using repeated treatment of combined siRNAs. The success of single versus combination siRNA treatments were also confirmed using a stable and persistently infected HCV cell culture system. The clearance of HCV in the infected culture was examined after repeated single or combination siRNA treatments by examining core protein expression by immunostaining. The number of cells expressing core protein in the persistently infected cells gradually decreased over time. These cells remained negative for core protein expression after 3 rounds of treatment using a combination of 2 siRNAs (si321+si359) (FIG. 4E). Treatment with a single siRNA was not effective to inhibit HCV infection. These cell culture studies indicate that rapid inhibition of HCV can be achieved by repeated treatment using 2 siRNAs complexed to or encapsulated in nanosomes.

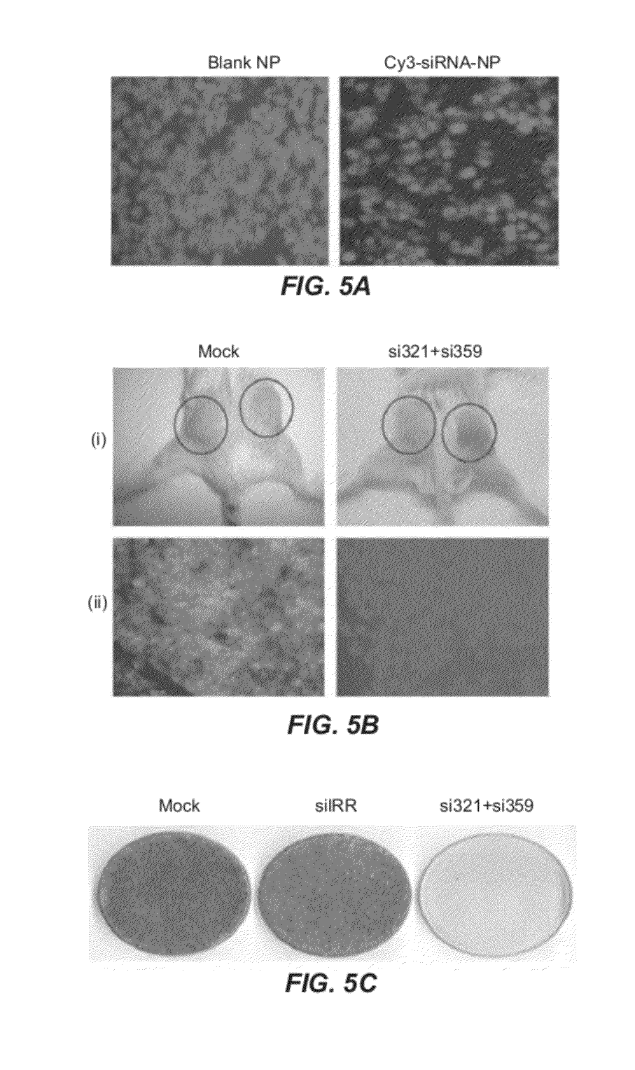

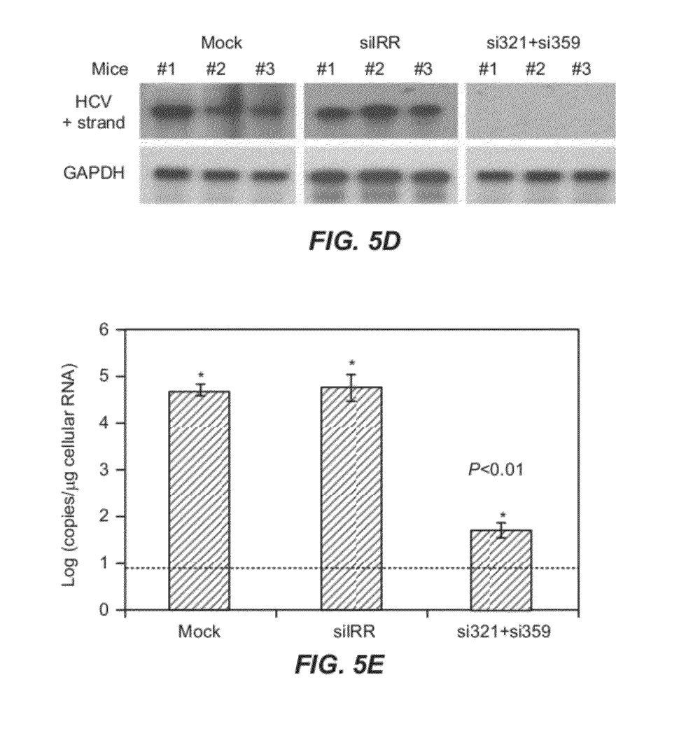

In vivo efficacy of siRNA-nanosomes in a subcutaneous tumor xenograft model: The antiviral effect of the combination siRNA treatment was validated in vivo using a tumor xenograft model for HCV in NOD/SCID mice that was developed in our laboratory. Hazari et al., World J Gastroenterol 17:300-312 (2011). Previous work indicated that 5 mg/kg siRNA is sufficient to achieve effective knockdown of the target gene in a mouse tumor model. Kim et al., J Hepatol 50:479-488 (2009); Watanabe et al., J Hepatol 47:744-750 (2007); Kim et al., Virus Res 122:1-10 (2006). Therefore, this dose was selected to examine in vivo efficacy of siRNA-nanoparticle treatment in a subcutaneous xenograft tumor model using the S3-GFP replicon. Cy3-labeled siRNA-nanosome complex in a 100 μl volume was injected into the area of the subcutaneously formed hepatocellular carcinoma (HCC) tumor. Intracellular uptake of siRNA was examined 24 hours post-administration in frozen tumor xenograft sections. The majority of the tumor cells took up Cy3-labeled siRNA (FIG. 5A). The siRNA-nanoparticle complexes were injected peritumorally 6 times every other day. The tumor size was the same between the groups that received nanosomes containing HCV-specific siRNA and unrelated siRNA targeted to EBV. All of the HCV siRNA-treated animals were negative for GFP expression in the tumor, whereas high expression of GFP was seen in the tumors that were injected with mock or control siRNA (FIG. 5B). The replication of HCV in the tumor was measured by culturing the tumor cells in a medium supplemented with G-418 (1 mg/ml). Tumor cells supporting HCV replication grew and formed distinct cell colonies in the presence of G-418, whereas cells lacking HCV did not. Results of this assay indicated that HCV-specific siRNA-nanosome complexes (si321+si359) effectively inhibited HCV RNA replication, compared to mock- or control siRNA-treated mice (FIG. 5C). The antiviral effect of siRNA-nanosome treatment on intracellular HCV RNA between different treatment groups was examined by ribonuclease protection assay (RPA) (FIG. 5D) and quantified by RT-qPCR (FIG. 5E). These results indicated that the combination of si321 and si359 considerably inhibited HCV replication in the subcutaneous tumor xenograft. The level of GAPDH mRNA remained the same throughout the treatment, demonstrating the specificity of the siRNA for HCV.

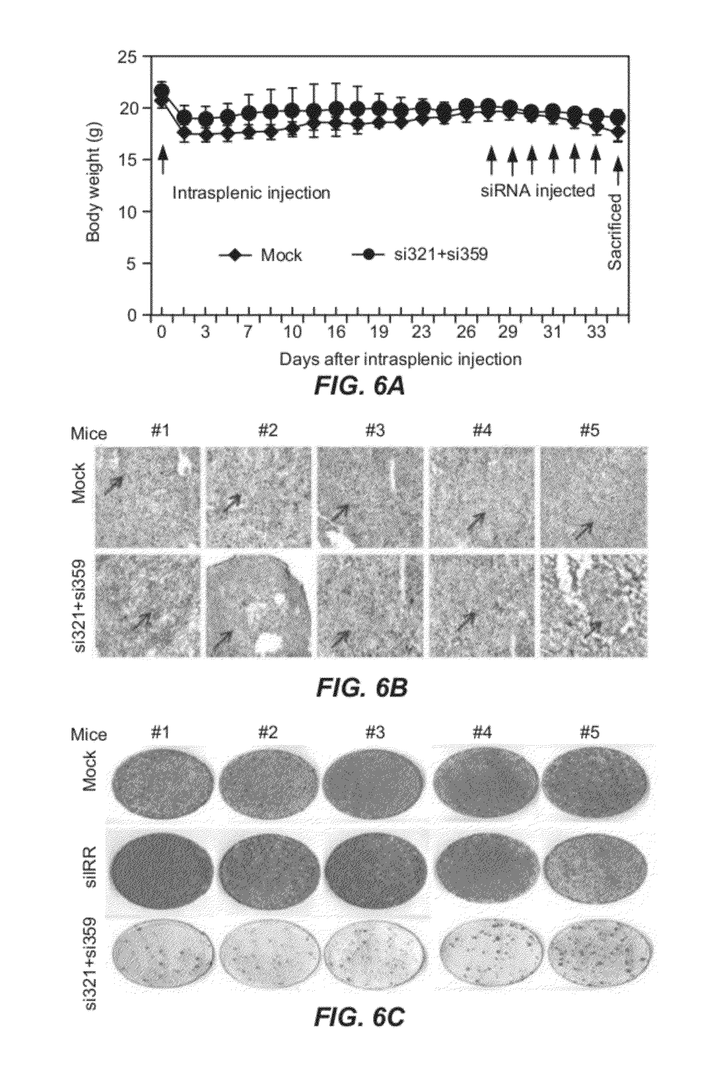

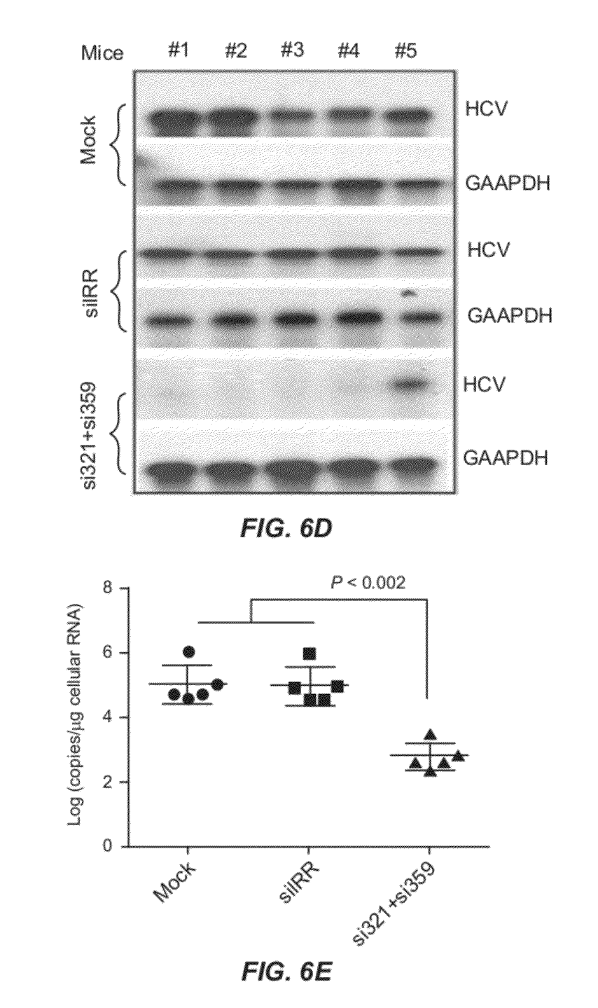

Systemic administration of siRNA-nanosome complex inhibits HCV replication in liver: We next determined whether replication of HCV in the liver can be inhibited after systemic delivery of siRNA-nanosome complex using a liver HCC xenograft mouse model. A total of 3 groups of 5 mice each were used. One group received combination treatment of si321 and si359. Another 2 control groups received systemic administration of nanosome with or without an irrelevant siRNA against EBNA1. Mice received 6 injections using 100 μl siRNA-nanosome at a dose of 5 mg/Kg body weight through their tail vein every day. Mice treated with the siRNA-nanosome formulation were healthy and survived to the end of the experiment. Body weights between untreated and siRNA-treated groups were comparable, which indicated that there was no adverse effect of siRNA-nanosome treatment (FIG. 6A). A histological examination of siRNA-treated and untreated animals revealed that there were a comparable number of intra-hepatic HCC cells, as shown by hematoxylin and eosin (H&E) staining (FIG. 6B). There was no evidence of hepatic toxicity found in the formalin-fixed tissue sections after H&E staining. There was a significantly lower number of G-418-resistant tumor cell colonies in the si321+si359-treated animals compared to mock- or control siRNA-treated groups (P<0.001), which indicated that siRNA treatment efficiently blocked HCV replication in the liver tumors (FIG. 6C). Inhibition of HCV replication was confirmed by measuring HCV RNA levels using RPA. Mice treated with siRNA-nanosome formulation had undetectable levels of HCV RNA, except for one mouse (FIG. 6D). Mice that received mock nanosome formulation or irrelevant siRNA did not show an inhibition of HCV replication Inhibition of HCV replication was further confirmed by measuring HCV RNA levels by RT-qPCR. The HCV RNA levels were significantly (P=0.002) reduced in the combination siRNA-treated mice (FIG. 6E).

We then clarified whether the lack of a complete elimination of HCV replication in the liver tumors was due to the emergence of escape mutants or an inadequate supply of siRNA in the tumor cells. For this purpose, HCV sequence analysis of three replicon colonies from each animal was performed. The sequences matched 100% with the wild type replicon. These findings suggest that the residual colonies that appeared in the siRNA-treated tumor cells were not due to the appearance of escape mutants. The incomplete clearance of HCV replication in the tumor cells was due to an insufficient supply of siRNAs to the tumor cells. Optimizing the dose of siRNA for an extended time should eliminate HCV replication in the tumor completely. In summary, these results suggest that effective inhibition of HCV replication in the liver can be achieved by systemic administration of siRNA-nanosome complexes.

Systemic administration of siRNA-nanosome complex is not toxic to BALB/c mice: The toxicity of multiple injections of siRNA-nanosome formulation was examined using 35 BALB/c mice by assessing overall body weight loss, serum enzyme levels (ALT, AST), and histopathology of different organs. Mice were injected with 100 μl siRNA-nanosome complex through the tail vein at a dose of 5 mg/kg body weight every other day and sacrificed at 0, 24, and 48 hours and 1 week after injection. Five BALB/c mice were used in each group. The body weights of the 5 mice after injection with siRNA-nanosome complex or saline alone showed no significant changes in 1 week (FIG. 7A). Serum enzyme levels (ALT and AST) remained within the normal range for BALB/c mice when measured at 0, 4, and 24 hours and 1 week (FIGS. 7B and 7C). The changes in ALT and AST expression between different experimental groups are not statistically significant, indicating that repeated systemic administration of siRNA-nanosome formulation did not cause liver toxicity. H&E-stained tissue sections of lung, heart, liver, spleen, and kidney were examined by two pathologists who did not have knowledge of the treatment status of the samples for evidence of potential cell necrosis due to toxicity, inflammatory cell infiltration, ballooning degeneration, and mitosis due to siRNA-nanosome formulation injection. There were no noticeable histological changes between the control and treatment groups (FIG. 7D). There were no specific liver histology alterations in BALB/c mice due to nanoparticle administration observed at untreated or 24 hours or 7 days after siRNA-nanosome injection. We also examined the histology of HCC and surrounding non-tumor liver of SCID/NOD mice after 6 injection of control siRNA which show no evidence of liver toxicity (FIG. 7E).

### Example 3

This Example discusses the results set forth above.

The studies set forth herein provide proof-of-principle for an intracellular therapeutic approach to inhibit chronic, persistent HCV infection through the systemic delivery of siRNA-lipid nanoparticles. Silencing of viral or cellular genes by siRNA has become a standard procedure in many research laboratories. The use of siRNA-mediated gene silencing in the treatment of human disease has been limited by the lack of an efficient siRNA in vivo delivery system. The improvements to this technology set forth herein allow efficient delivery of siRNA in vivo and facilitate therapeutic use in humans. Intracellular delivery of siRNA is a major challenge due to the stability of siRNA in the serum and inability of large, negatively charged molecules to cross the cell membrane. The cationic lipid DOTAP is suitable because its net positive change enhances complex formation with polyanionic nucleic acids such as siRNA and facilitates interaction with the cell membrane. In this study, cationic lipid (DOTAP)-based nanometer-sized lipid nanoparticles called nanosomes were formulated. Multiple siRNAs targeting different locations of the HCV 5′UTR were chemically synthesized and incorporated into the lipid nanoparticle using protamine sulfate. The success of siRNA treatment of chronic HCV infection in the liver requires the siRNA-nanosome complex particle size to be small enough to prevent clogging the capillaries to pass the endothelial barrier to reach the infected hepatocytes. (Fumoto et al., Pharm. Res 20:1452-1459 (2003); Higuchi et al., Biol. Pharm. Bull 29:1521-1523 (2006); Braet and Wisse, Comp Hepatol 1:1 (2002)). Therefore, the formulation was sonicated to create smaller particles. The zeta potential of the lipid nanoparticles was optimized by changing the lipid-to-siRNA ratio to improve siRNA delivery to hepatocytes. The siRNA delivered by nanosome is stable and functionally active in the cytoplasm, and repeated treatment is well tolerated without any liver toxicity.

A particular concern with the siRNA-nanosome complex-based approach is the possibility of in vivo toxicity after systemic delivery. Toxicity studies were conducted after systemic administration of siRNA-nanosome formulation to BALB/c mice. We show that systemic administration siRNA-nanosome formulation at a dose of 5 mg/kg body weight is well tolerated in a BALB/c mouse model without elevation of liver enzymes or evidence of liver toxicity. The siRNA-nanosome formulation did not activate the intracellular interferon system, indicating that delivery of siRNA by nanosomes represents a viable approach to inhibit HCV replication. We have also recently shown that the siRNA-nanosome formulation can be stored for more than 3 months in lyophilized form without significant loss of antiviral activity. Kundu et al., Int. J Pharm 423:525-534 (2012).

An obvious challenge in treating chronic HCV infection with a siRNA-based antiviral strategy is minimizing the development of escape mutant viruses. Therefore, we tested whether or not the siRNA-based antiviral strategy could be applied to silence HCV replication using an interferon-resistant replicon and an infectious HCV cell culture system. The clinical use of the siRNA-based antiviral strategy against HCV is dependent on the selection of an appropriate target within the viral RNA genome that can be used for all viral strains. Clinical HCV strains in humans have been classified into 7 major types and numerous subtypes differing by approximately 31-33% and 20-25% of their genome sequences, respectively. Li et al., Virology 421:222-234 (2011); Sagan et al., Chem Biol 17:515-527 (2010). There are more nucleotide variations in the coding region than the non-coding region, making it difficult to develop consensus siRNA targets in the coding areas that can be used for all HCV strains. The 5′UTR acts as an internal ribosome entry site for protein translation, the activity of which is dependent on RNA secondary structure. This region does not tolerate nucleotide changes and is highly conserved among all HCV genotypes. Targeting this region for RNAi reduces the mutational freedom and minimize the development of escape mutants. However, other studies indicate that escape mutants also appear when the highly conserved regions of the HIV genome is targeted with an siRNA-based antiviral approach. (Westerhout et al., Nucleic Acids Res 33:796-804; Nevot et al., J Mol Biol 413:84-96 (2011); Boden et al., J Virol 77:11531-11535 (2003); Luo and Chang, Biochem Biophys Res Commun 318:303-310 (2004); Yoshinari et al., Nucleic Acids Res 32:691-699 (2004); Shubert et al., J Mol Biol 348:883-893 (2005); Westerhout and Berkhout, Nucleic Acids Res 35:4322-4330 (2007)).

Multiple siRNAs targeting stem-loops III and IV of the highly conserved 5′UTR of the HCV genome were tested for their ability to inhibit HCV replication in cell culture relative to irrelevant control siRNAs. Antiviral efficacies of the siRNAs targeting stem-loop IV varied significantly, which may be because sequences in stem-loop IV have secondary structures that reduce accessibility for RNA silencing. Another potential explanation may be that cellular and ribosomal proteins that have been reported to bind to the stem-loop IV region may interfere with siRNA binding. Sarnow, J Virol 77:2801-2806 (2003). We showed that treatments using a single siRNA lead to the development of escape mutant viruses in a replicon cell line and infected cell culture. The appearance of the escape mutant virus was abolished when 2 siRNAs targeted to different locations of the 5′UTR of the HCV genome were used. We showed that 3 combinations of 2 siRNAs led to rapid inhibition of HCV in the replicon as well as in the infectious cell culture model. The level of HCV RNA remained below the detection threshold in the infected cells after 3 passages, whereas the HCV RNA was detectable in the infected culture when treated with a single siRNA over 5 passages. We showed that 6 siRNAs targeted to the 5′UTR can be used in combination treatments to silence HCV infection. A recent report claimed that combination siRNA treatment may reduce antiviral efficacy because of incomplete dicer processing of small hairpin RNAs. ter Brake et al., Mol Ther 16:557-564 (2008). We did not find any evidence of low antiviral activity when 2 siRNAs targeted to different locations in the same HCV RNA molecule were combined.

Significant progress has been made in the siRNA delivery system using novel approaches in various disease models, such as cancer and infectious diseases, including HIV. Burbett and Rossi, Chem Biol 19:60-71 (2012); Kanasty et al., Mol Ther 20:513-524 (2012). Several investigators have demonstrated cationic liposome-based siRNA delivery to the liver to inhibit HCV gene expression in vivo. Kim et al., J Hepatol 50:479-488 (2009); Watanabe et al., J Hepatol 47:744-750 (2007); Kim et al., Virus Res 122:1-10 (2006). We performed studies to show that an siRNA-based antiviral strategy can be successfully used to inhibit HCV replication in the liver. The results show that 6 injections of siRNA-nanosome complexes lead to significant inhibition of viral RNA replication in HCC tumor xenografts. These results indicate that the siRNA-nanosome delivery system is a therapeutic strategy for the treatment of chronic HCV infection. The combinatorial use of two siRNA targeting different locations of the HCV 5′UTR can be utilized in the treatment of chronic HCV infections, particularly those that are refractory to standard IFN-α, ribavirin and protease inhibitor-based triple combination therapy.

### Example 4

This Example relates to studies in which HCV infected cells were treated with siRNA-nanosomes containing the siRNAs set forth in Table 1.

siRNA-nanosome treatment in the replicon model. R4-GFP cells (2×10⁵ cells/well) were treated with nanosomes containing 13 HCV specific and one control siRNA (an irrelevant siRNA, “siIRR,” targeted to Epstein-Barr virus nuclear antigen). The antiviral effect of the single siRNAs and of combinations of siRNAs was first examined by detection of GFP fluorescence, which was subsequently quantified by flow cytometry.

siRNA-nanosome inhibition of HCV replication: Inhibition of HCV replication in infected cell cultures after siRNA-nanosome treatment was determined by performing a multi-cycle infectivity assay. To screen the antiviral efficacy of the 13 siRNAs, 100 pmole of an individual siRNA were transfected and, after 72 hours, the luciferase activity was measured. Complete clearance or the development of an escape mutant virus was assessed by following the infected culture for 80-days after four treatments. HCV replication was measured by luciferase assay.

TABLE 1
Sequences of siRNAs targeted to the 5′ UTR of HCV

| | | | SEQ |
|---|---|---|---|
| No. | siRNA | siRNA sequences | ID NO: |
| 1 | si74 | 5′-GCGCCUAGCCAUGGCGUUATT-3′ | 1 |
| | | 3′-TTCGCGGAUCGGUACCGCAAU-5′ | 2 |
| 2 | si173 | 5′-UUGCCGGGAAGACUGGGUCTT-3′ | 3 |
| | | 3′-TTAACGGCCCUUCUGACCCAG-5′ | 4 |
| 3 | si206 | 5′-CCCACUCUAUGCCCGGCCATT-3′ | 5 |
| | | 3′-TTGGGUGAGAUACGGGCCGGU-5′ | 6 |
| 4 | si244 | 5′-GACUGCUAGCCGAGUAGCGTT-3′ | 7 |
| | | 3′-TTCUGACGAUCGGCUCAUCGC-5′ | 8 |
| 5 | si261 | 5′-CGUUGGGUUGCGAAAGGCCTT-3′ | 9 |
| | | 3′-TTGCAACCCAACGCUUUCCGG-5′ | 10 |
| 6 | si279 | 5′-CUUGUGGUACUGCCUGAUATT-3′ | 11 |
| | | 3′-TTGAACACCAUGACGGACUAU-5′ | 12 |
| 7 | si298 | 5′-GGGCGCUUGCGAGUGCCCCTT-3′ | 13 |
| | | 3′-TTCCCGCGAACGCUCACGGGG-5′ | 14 |
| 8 | si315 | 5′-CCGGGAGGUCUCGUAGACCTT-3′ | 15 |
| | | 3′-TTGGCCCUCCAGAGCAUCUGG-5′ | 16 |
| 9 | si321 | 5′-GGUCUCGUAGACCGUGCACTT-3′ | 17 |
| | | 3′-TTCCAGAGCAUCUGGCACGUG-5′ | 18 |
| 10 | si333 | 5′-CGUGCACCAUGAGCACAAATT-3′ | 19 |
| | | 3′-TTGCACGUGGUACUCGUGUUU-5′ | 20 |
| 11 | si351 | 5′-AUCCUAAACCUCAAAGAAATT-3′ | 21 |
| | | 3′-TTUAGGAUUUGGAGUUUCUUU-5′ | 22 |
| 12 | si359 | 5′-CCUCAAAGAAAAACCAAAATT-3′ | 23 |
| | | 3′-TTGGAGUUUCUUUUUGGUUUU-5′ | 24 |
| 13 | si369 | 5′-AAACCAAAAGAAACACCAATT-3′ | 25 |
| | | 3′-TTUUUGGUUUUCUUUGUGGUU-5′ | 26 |
| 14 | siIRR | 5′-ACGUCCAAGUUCGAUUGGCTT-3′ | 27 |
| | | 3′-TTUGCAGGUUCAAGCUAACCG-5′ | 28 |

### Example 5

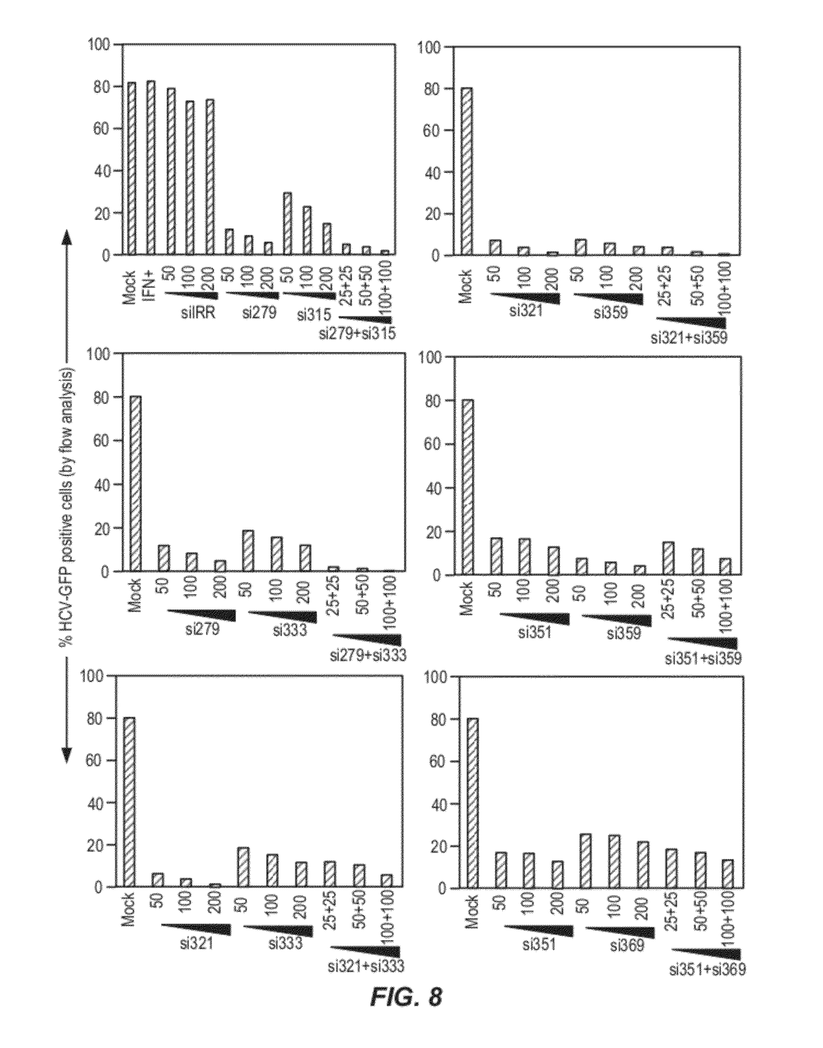

FIG. 8 sets forth the results of studies of the inhibition of HCV using single siRNA-nanosomes using siRNAs set forth in Table 1, and combinations of siRNA-nanosomes using siRNAs set forth in Table 1. Different siRNAs were introduced by nanosomes into HCV-infected cells and the antiviral effects after two consecutive treatments were assessed by measuring the HCV-GFP positive cells by flow analysis. Combinations of si279+si315, si321+si359, and si279+si333 were highly effective in inhibiting HCV replication.

### Example 6

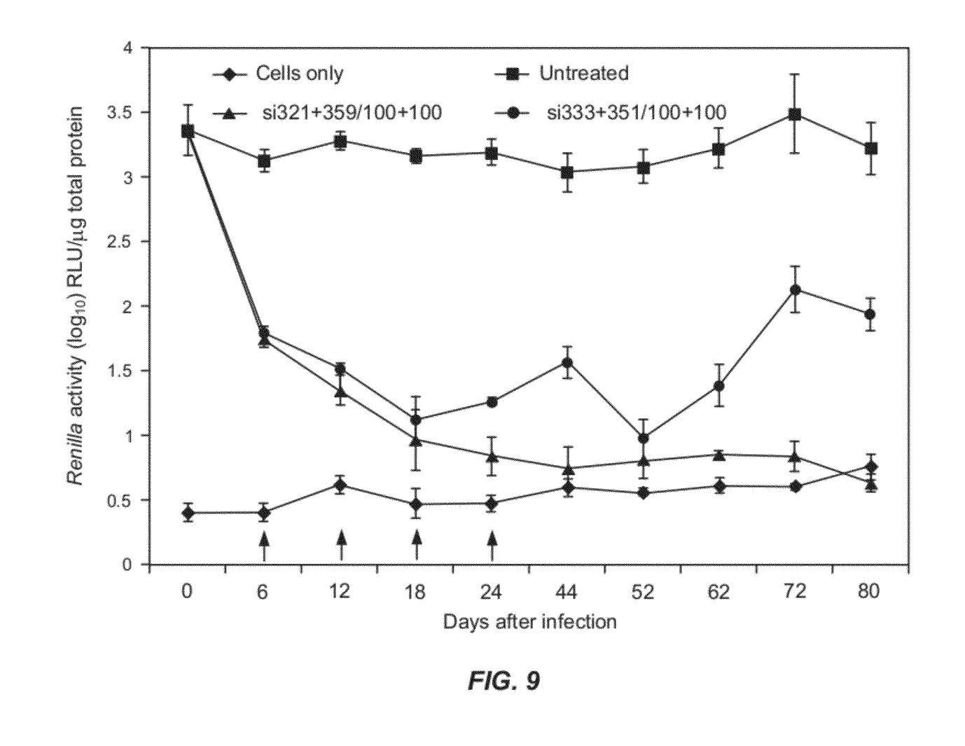

Studies were conducted comparing the ability of two different combinations of siRNAs to inhibit HCV replication in infected cells. As shown in FIG. 9, persistently infected cells were treated at days 6, 12, 18, and 24 after infection with (a) a combination of si321 and si359, at a concentration of 100 pmol each, or (b) a combination of si333 and si351, at a concentration of 100 pmol each. Cultures were followed for 80 days after infection. As shown in FIG. 9, 4 treatments with the combination of si321 and si359 resulted in HCV replication, as shown by Renilla luciferase activity in RLU, dropping to approximately the level of uninfected cells, indicating a complete inhibition of HCV activity in the cells treated with this combination of siRNAs. In contrast, cells treated with the combination of si333 and si351 showed a marked decline in HCV activity over the first three treatments, but then showed a rebound in HCV activity, indicating continued HCV replication in the cells. Accordingly, in this study, the si321+si359 combination was able to completely inhibit the replication of HCV in the cells, but the si333+si351 combination was not.

### Example 7

This Example sets forth materials and methods used in the studies reported below.

DOTAP and cholesterol were purchased from Avanti Polar-lipids Inc. (Birmingham, Ala.). Protamine sulfate salt Grade X (PS), trehalose dihydrate and HPLC grade chloroform were obtained from Sigma Chemical Co. (St. Louis, Mo.). Functional siRNA (5′- CCU CAA AGA AAA ACC AAA ATT -3′) (SEQ ID NO:42) and control siRNA (5′-GCG CCU AGC CAU GGC GUU ATT -3′) (SEQ ID NO:43) and silencer cy3-labeled GAPDH siRNA were designed and purchased from Applied Biosystems (Austin, Tex.). Silencer GAPDH siRNA (Cat. No. AM4631), fetal bovine serum albumin (BSA), Dulbecco's modified Eagle's medium (DMEM), penicillin/streptomycin and lipofectamine 2000 were purchased from Invitrogen Corp. (Carlsbad, Calif.). The RiboGreen® assay kit was supplied by Molecular Probes (Eugene, Oreg.). The anti-rabbit GAPDH antibody, anti-rabbit β-actin antibody and anti-rabbit IgG HRP labeled secondary antibody were purchased from Cell Signaling Technology (Danver, Mass.). IFN-1 (Interferon-1) was purchased from Schering Corporation (New Jersey, N.J.). All other reagents were of analytical grade and were supplied by Sigma Chemical Co. (St. Louis, Mo.).

Preparation of nanosomes: Nanosomes were prepared by an EmulsiFlex-B3 high pressure homogenizer (HPH) (Avestin Inc., Ottawa, Canada). In brief, the nanosomes were prepared from a mixture of two lipids; cholesterol and DOTAP, at the molar ratio of 1:1. The lipids (containing 50 mg DOTAP and 26.7 mg cholesterol) were dissolved in 15 ml HPLC-grade chloroform in a round bottom flask and then dried under nitrogen gas and overnight vacuum. The resulting films of the lipids were hydrated in de-ionized water to give a final concentration of 20 mM. The lipid dispersions were warmed and mixed at 50° C. for 45 minutes by rotation, followed by warming again at 35° C. for another 10 minutes. The resultant dispersion was stored at room temperature for 3 hours before it was transferred into a scintillation vial and warmed again at 50° C. for 10 minutes. The final lipid dispersion was homogenized by using HPH at 20,000 PSI for 5 cycles. Each time, 2.5 ml of lipid dispersion was subjected to homogenization and the resultant nanosomes were collected in another scintillation vial. The nanosomes were kept at room temperature for 1 hour prior to overnight storage at 4° C.

Preparation of siRNA loaded nanosomes: Five different siRNA loaded nanosome formulations (F1, F2, F3, F4, and F5) were prepared by using homogenized nanosomes, protamine sulfate, trehalose and siRNA. The composition of the different formulations and siRNA amounts used for the experiments is listed in Table 1. In brief, freshly prepared protamine sulfate solution in DEPC-treated water was added drop-wise to an aqueous solution of siRNA (10 pmol/μl) while vortexing the solution at a moderate speed. siRNA and protamine sulfate condensation was performed at room temperature by incubation of the mixture for 40 minutes. Following the siRNA-protamine sulfate condensation, the pre-warmed nanosomes (20 mM) were added to the mixture. The final preparations were mixed rapidly by pipetting up and down thirty times. Finally, freshly prepared trehalose solution in DEPC-treated water (0.0301 nM) was added to the nanosome formulations, followed by vortexing four times to allow thorough mixing of the siRNA nanosomes with trehalose.

To study the effect of sonication on the siRNA nanosome formulations, a modified F4 containing cy3-GAPDH siRNA was used instead of the functional siRNA. In this experiment, after preparing a bulk amount of modified F4, it was aliquoted into a set of Eppendorf tubes (B1-B9), each containing 20 μg siRNA encapsulated nanosomes in 60 μl total volume (Table 3). These preparations were subsequently diluted to 1 ml by adding DEPC-treated water. Each formulation was sonicated in ice cold water in a water bath for a specific time period (Table 3). The sonicated formulations were always freshly prepared for different experiments.

Measurement of particle size, particle morphology and zeta potential: The mean particle size of the siRNA nanosomes (B1-B9) was determined by dynamic laser light scattering method at room temperature by using a Delsa Nano C Particle Analyzer (Beckman Coulter Inc., Fullerton, Calif.). The particle size was reported as the mean±standard deviation (n=4).

The morphology of different sonicated nanosomal particles was examined by a cryo-Transmission Electron Microscope (cryo-TEM; JEOL 2010, Peabody, Mass.). A 6 μl drop of each formulation (B1, B4, B6 and B8) was placed on a holey carbon grid and rapidly vitrified in liquid ethane. The sample was then transferred under the protection of liquid nitrogen to the cryo-TEM sample holder and inserted into the cryo-TEM. The temperature of the sample grids was maintained at −175° C. during the course of imaging.

Analysis of the charge density on the sonicated siRNA loaded nanosomes was performed by examining their zeta potential with a Malvern Zetasizer 2000 (Malvern Instruments, Malvern, UK). The system was initially calibrated with standards. All experimental samples were prepared in 1 mM KCl and five measurements times.

Measurement of siRNA encapsulation efficiency in different sonicated formulations: The amount of siRNA, either complexed or encapsulated, was determined for different sonicated formulations. The efficiency of encapsulation was calculated by comparing the total amount of siRNA measured in the sample with the actual amount of siRNA added to the sample. Briefly, after preparation, the samples (B1-B9) were centrifuged at 14,000 rpm (Allegra Centrifuge, Beckman Coulter Inc., Fullerton, Calif.) for 15 minutes at 4° C. Supernatants containing the free siRNA were separated from the pellets. Five hundred μl of a 1% sodium dodecyl sulfate (SDS) solution was added to the pellets, and to the supernatants. Samples were then incubated at 37° C. for 18 hours with gentle agitation (50 rpm). The siRNA amount from both supernatants and pellets was measured by using RiboGreen® assay, following the manufacturer's protocol. The results were reported as the mean±standard deviation (n=4).

Agarose gel electrophoresis: The structural integrity of encapsulated siRNA in different sonicated formulations was assessed by agarose gel electrophoresis. In brief, after extracting siRNA by using 500 μl of 1% SDS, the solution was mixed with an equal volume of phenol and chloroform (1:1) and then vortexed for 1 minute using the Vortex Genie Mixer (Scientific Industries Inc., Bohemia, N.Y.) and stored at room temperature for 5 minutes. The resultant mixture was centrifuged at 11,000 rpm for 15 minutes and the aqueous layer was collected. The siRNA present in the aqueous layer was mixed with 1 ml isopropanol and kept at −80° C. overnight. The mixture was again centrifuged at 11,000 rpm for 30 minutes at 4° C. and the pellets were collected. Finally, the siRNA was precipitated and purified by adding 20 μl 2M sodium acetate and absolute ethanol (100% v/v), centrifuging at 11000 rpm for 30 minutes at 4° C., and subsequently run on a 3% agarose gel.

Measurement of intracellular localization of siRNA by confocal microscope: For measuring intracellular localization of siRNA, Huh-7 cells were cultured in 8-well chamber slides (Electron Microcopy Sciences, Hatfield, Pa.) for 24 hours and transfected with B6 encapsulating cy3-GAPDH siRNA for different time periods (i.e. 30 minutes, 2 hours and 12 hours, respectively). The endosomes and lysosomes were stained by incubating those B6 transfected cells with LysoTraker® Green DND-26 (Molecular Probes, Life Technologies, Grand Island, N.Y.) and the cells were fixed with 4% paraformaldehyde solution after several washings with phosphate buffered saline. Nuclei were also stained with To-Pro®-3 nuclear marker (Molecular Probes) and observed with Leica TCS SP2 confocal microscope equipped with three lasers (Leica Microsystems, Exton, Pa.).

Cell culture and cell lines: Huh-7 cells and R4GFP HCV (+) replicon cells were used. The cells were routinely cultured and expanded at 37° C. in DMEM, supplemented with 2 mM 1-glutamine, nonessential amino acids, 100 U/ml of penicillin, 100 μg/ml of streptomycin and 5% fetal bovine serum in an atmosphere of 5% CO₂.

In vitro functional assessment of HCV inhibition by different siRNA nanosomes (F1-F5): GFP expression: In vitro transfection efficiency and inhibition of HCV by different formulations (F1-F5) were monitored for three consecutive doses by qualitative (GFP expression) and quantitative (FACS) analysis. Briefly, 2×10⁵ R4GFP cells were cultured in 6-well tissue culture plates (TCPs) for 24 hours. The media was replenished by 1 ml fresh media and the cells were transfected with lipofectamine, or with siRNA nanosomes encapsulating 100 pmol siRNA (i.e. 100 nM siRNA based on 1 ml cell culture media). After 4 hours, an additional 1 ml of fresh media was added and cell culture was continued for 48 hours. The cells were then scraped by using trypsin-EDTA and re-suspended into 1 ml of media. The cell number of control experiments (i.e. mock) was counted, and the suspension media containing 2×10⁵ R4GFP cells in the control experiment was measured. This amount (i.e. volume) of cell suspension media was then taken to re-plate cells for each experimental condition and cultured again for 48 hours. After this time period, the cells were again transfected a second time with the same nanosome formulations, or with lipofectamine entrapping the same amount of siRNA for another 48 hours. This procedure was followed again to re-plate cells for a third dose of transfection. Simultaneously, in another well, cells were treated with 1000 IU/ml IFN for all three dose points. Following the third dose, the GFP expression was monitored by using a fluorescence microscope (Olympus America, Inc., NY) and photographs were captured at 10× magnification.

FACS analysis: After capturing GFP images by fluorescent microscope, flow cytometric analysis (FACS) (BD Biosciences, San Jose, Calif.) was performed to quantify the GFP-positive cells post-treatment.

Ribonuclease Protection Assay (RPA): A positive strand of human GAPDH mRNA level was detected by RPA. Briefly, Huh-7 cells were transfected with different formulations for two consecutive doses as stated above. Subsequently, total RNA was isolated from the transfected cells by the GITC method and subjected to RPA using a probe targeted to human GAPDH mRNA. A linearized plasmid pTRI-GAPDH-human antisense control template was used to prepare the probe to detect GAPDH mRNA by using Sp6 RNA polymerase (Ambion Inc., Austin, Tex.).

Measurement of cell viability: The toxicity of different siRNA nanosomes (F1-F5) and lipofectamine transfection was determined on R4GFP replicon cells by the MTT assay according to the manufacturer's protocol (Sigma Chemical Co., MO). The cells (2×10⁴) were cultured on 24-well TCPs for 24 hours followed by transfection with different siRNA nanosomes (10 nM), or with lipofectamine for another 48 hours. Simultaneously, 2×10⁵ R4GFPcells were cultured in a 6-well plate and transfected with 100 nM siRNA formulations, or with lipofectamine for 48 hours. The cells from 6-well plates were then scraped by trypsin-EDTA and re-suspended into 1 ml media. The suspension media of control experiments (i.e. mock) that contained 2×10⁴ and 2×10⁵ R4GFP cells was measured and this volume of suspension media was taken to re-plate cells for each experimental condition in 24- and 6-well plates, respectively. After a 48 hour incubation period, the cells in the 24- and 6-well plates were transfected a second time with various formulations or with lipofectamine entrapping the same amount of siRNA (i.e. 10 nM for 24-well plates and 100 nM for 6-well plates) for another 48 hours. For the experiment with the third dose transfection, the same procedure was followed for the cell scraping and counting from 6-well plates to replate them in 24-well plates. Finally, the MTT assay was performed after completion of 1^{st}, 2^{nd} and 3^{rd} dose in 24-well plates by incubating MTT solution at 37° C. for 3 hours, and the cell viability was measured by reading the absorbance at 570 nm.

Delivery of cy3-GAPDH siRNA: For optimizing siRNA amount, another set of formulations was also prepared by adding different amount of siRNA (cy3-GAPDH siRNA) into the siRNA nanosomes while keeping the amount of liposomes, protamine sulphate and trehalose unchanged (i.e. liposome: PS: trehalose=13.36:2:10). This set of formulations was prepared by following the same procedure already stated above. The formulations were used to transfect Huh-7 cells in 12-well TCPs for 24 hours and the transfection was observed at 10× magnification by using a fluorescence microscope and quantitatively measured by FACS analysis.

Experimental Animals: Female Balb/c mice (8-10 weeks old) were obtained from Charles River Laboratories (Wilmington, Mass.). The mice were housed at the Tulane Vivarium facility. This facility complies with the recommendations of the Guide for Care and Use of Laboratory Animals with respect to restraint, husbandry, surgical procedures, feed and fluid regulation, and veterinary care. All experiments performed on the animals were in accordance with, and approved by the Tulane University Animal Care and Use Committee (IACUC).

In vivo delivery of siRNA nanosomes in mice: The Cy3-GAPDH siRNA encapsulated nanosomes were administered via the intraperitoneal route (IP), whereas the functional GAPDH siRNA nanosomes were administered via both IP and intravenous routes (IV) to Balb/c mice. For cy3-GAPDH siRNA delivery, 50 μg siRNA (at 2.5 mg/kg body weight) was used to prepare nanosomes (B 1 and B6), and for GAPDH siRNA delivery, 100 μg siRNA (at 5.0 mg/kg body weight) was used to prepare nanosomes (B1 and B6). Another blank nanosome formulation (BO) was also prepared without the siRNAs. The B0 and B6 formulations were exposed to 4 minutes sonication in ice cold water, whereas the B1 formulation was kept non-sonicated. After 6 hours of cy3-GAPDH siRNA delivery, the mice were sacrificed by euthanization and the organs (liver, lung, heart, kidney and spleen) were collected immediately and embedded in Tissue-Tek® OCT™ embedding compound (American Master Tek Scientific, Lodi, Calif.) and stored in dry ice. The tissue sections cut at 12 μm thickness were analyzed for cy3-GAPDH siRNA deposition in different organs by using a fluorescence microscope at 10× magnification. For the functional GAPDH siRNA delivery group, the siRNA nanosomes were injected for three consecutive doses over a period of three days. Finally, the mice were sacrificed by euthanization and the liver was collected and stored at −80° C.

Western Blot analysis: A small portion of each lobe of the liver tissue was separated, weighed and lysed by RIPA lysis buffer with proteinase and phosphatase inhibitors (1×PBS, 1% NP-40, 0.5% deoxycholate, 0.1% SDS, 50 μg/ml PMSF, 5 μg/ml aprotinin, 5 μg/ml leupeptin, 1 μg/ml pepstatin). The tissue lysates were cleared by centrifugation at 14000 rpm for 20 minutes, the supernatant was collected and an equal amount of proteins (7 μg) was run for Western blotting to detect GAPDH (1:2000 dilution) and β-actin (1:2000 dilution).

Histology and Immunocytochemistry: The histological features of the frozen section of liver tissue of Balb/C mice which were treated with control (B0), sonicated (B6) or non-sonicated (B1) formulations were examined by hematoxylin and eosin staining (H&E). Briefly, five-micrometer sections were cut from each tissue block and were examined by a routine H&E staining procedure. To demonstrate the knock down of GAPDH in the liver hepatocytes, the tissue sections were immunostained with an antibody against mouse GAPDH (1:100 dilution) (Cell Signaling Technology, Danver, Mass.). The immunereactivity of the GAPDH antibody was detected using the ABC detection kit (Vector Lab, Burlingame, Calif.) using a standard laboratory protocol. The counterstaining of the nucleus was done by CAT hematoxylin. The cytoplasmic expression of GAPDH (brown staining) of mouse hepatocytes in the liver sections was subsequently captured via microscope (Olympus IX 60, Germany) at 20× magnification.

Statistical analysis: The results were expressed as mean±standard deviation. Statistical significance between groups (i.e. F4 and lipofectamine transfection) during FACS analysis of HCV inhibition and MTT assay of cell viability was compared by the Student-Newman-Keul's nonparametric test, using GraphPad Prism 5 software. A p value of <0.05 was considered as evidence of a significant difference.

TABLE 2
Composition of different siRNA nanosomes for inhibition of
HCV replication.

| | | | Total | | | siRNA |
|---|---|---|---|---|---|---|
| Nanosome | | Chol- | Lipids | Functional | Control | concen- |
| Formulation | DOTAP | esterol | (μg) | siRNA | siRNA | tration* |
| F1 | 2.09 μg | 1.16 μg | 3.25 | 1.0 μg | — | 100 nm |
| F2 | 3.44 μg | 1.90 μg | 5.34 | ″ | — | ″ |
| F3 | 6.80 μg | 3.76 μg | 10.56 | ″ | — | ″ |
| F4 | 8.60 μg | 4.76 μg | 13.36 | ″ | — | ″ |
| F5 | 8.60 μg | 4.76 μg | 13.36 | — | 1.0 μg | ″ |

*siRNA concentration was determined based on 1 ml cell culture media.

Each formulation also contained protamine sulfate=2 μg and trehalose=10 μg.

### Example 8

This Example sets forth the results of studies using the materials and methods set forth in the previous Example.

Endosomal escape and siRNA release in the cell cytoplasm: Huh-7 cells were transfected with increasing concentrations of cy3-GAPDH siRNA nanosomes. Table 3 demonstrates the efficiency of delivery; the delivery of siRNA into the cell cytoplasm was observed by using as little as 0.74 nM siRNA. The FACS analysis data of the deposited particles further indicates that nearly all cells took up the particles at ≧7.4 nM of siRNA and the mean intensity of deposited siRNA into the cells gradually increased with the increase of siRNA addition to the nanosomes (Table 3). However, truly significant clearance of HCV from R4GFP HCV (+) cells by siRNA nanosomes was attained at 100 nM siRNA (i.e. functional siRNA), a concentration which was associated with minimal or no cytotoxicity. This observation was the rationale for using this specific amount of siRNA (100 nM) for encapsulation into the nanosomes. To gain further insight into siRNA delivery into the cytoplasm, the current study tracked siRNA localization, as well as endosome formation within the cells at different time periods. The siRNA nanosomes need to cross the cell plasma membrane and reach into the cytoplasm to initiate gene silencing activities. The siRNA should subsequently be released from the endosome/lysosome compartments. The results indicate that within 30 minutes of transfection, the siRNA nanosomes were associated with the plasma membrane, a few of them were engulfed into the endosomes, while the remaining particles were sitting on the plasma membrane. After 2 hours of cell transfection, a high percentage of cy3-lebeled siRNA nanosomes was found to be located in the cell cytoplasm. The LysoTraker™ also tracked endosomes in both the cytoplasmic and peripheral regions of the cells. When these two images were merged, it was noticed that most of the siRNA nanosomes were confined within the endosomes in the cell cytoplasm. The siRNA particles were yet to release from the endosome vesicles. After 12 hours of transfection, the particles (siRNA nanosomes) were observed to break into small pieces allowing release of siRNA, as evidenced by the presence of red colored smears throughout the cytoplasm. The endosomes did not appear to be holding any siRNA nanosomes after this 12 hour time period. This study therefore demonstrates that the siRNA nanosomes are taken up by the endosomes and then carried into the cell cytoplasm within 2 hours of transfection. Later, the siRNA is released from the endosomes within 12 hours of transfection and become available for functional gene silencing.

TABLE 3
Measurement of siRNA accumulation into Huh-7 cells by entrapping
different amounts of siRNA into siRNA-nanosomes.

| siRNA | siRNA accumulation into Huh-7 cells | |
|---|---|---|
| concentration | Percentage of cells | Mean intensity (×1000) |
| 0 nM | 0.0 | 0.0 |
| 0.74 nM | 24.4 ± 6.0 | 0.44 ± 0.008 |
| 3.7 nM | 93.8 ± 4.0 | 0.94 ± 0.07 |
| 7.4 nM | 99.4 ± 0.05 | 1.96 ± 0.10 |
| 37.0 nM | 100.0 ± 0.0 | 5.87 ± 0.24 |
| 74.0 nM | 100.0 ± 0.0 | 11.82 ± 0.13 |
| 148.0 nM | 100.0 ± 0.0 | 17.94 ± 0.36 |
| 222.0 nM | 99.86 ± 0.15 | 20.73 ± 1.20 |

Inhibition of HCV replication in cell culture model: The inhibition of HCV replication in the in vitro cell culture study was monitored for three consecutive doses of siRNA nanosomes and compared with lipofectamine transfection. Of the formulations F1 to F4, significantly high HCV inhibition was observed in both F3 and F4. F1 and F2 were not as effective as F3 and F4, as several GFP positive cells were evident in these two siRNA nanosomes treated cells. The results were also confirmed from the quantitative FACS analysis of GFP expression. The inhibition of HCV replicon by these siRNA nanosomes was ranked as follows: F4>F3>F2>F1. The clearance of HCV by either F3 or F4 was significantly higher than by F1 or F2 at different dose points, though the inhibition of HCV replication from 2^{nd} to 3^{rd} dose for F3 and F4 was not as significant as observed with F1 and F2. On the other hand, when the knock-down efficiency of F4 was compared with lipofectamine, a more significant inhibition of HCV replication was observed for F4 compared to lipofectamine at different dose points (i.e. ˜87% vs. ˜57% for 1^{st} dose, ˜91% vs. ˜64% for 2^{nd} dose and ˜91% vs. ˜80% for 3^{rd} dose) (p<0.0001). This study thus indicates that a lipid to siRNA ratio ≧10.56:1 is more effective in its ability to inhibit HCV replication, than the corresponding ratios ≦3.25:1. No inhibition of virus replication was observed in the control siRNA nanosomes (F5) or with IFN-1.

Cell viability: The viability of R4GFP cells during the process of clearing HCV replication by different siRNA nanosomes was also monitored for 3 consecutive doses and the results were again compared with lipofectamine transfection. The results indicate that F1 and F2 were able to maintain cell viability ˜100% during the 1^{st} and 2^{nd} dose, following which a slight decline of cell viability was observed after the 3^{rd} dose. When the lipid amount was comparatively higher, as in F3 and F4, the cell viability was 2^{nd} determined to be ˜90%, ˜86%, ˜86% for F3 and ˜89%, ˜81%, ˜79% for F4 after 1^{st}, and 3^{rd} doses, respectively. In the case of lipofectamine transfection, the cell viability was shown to be ˜83%, ˜70% and ˜68% after 1^{st}, 2^{nd} and 3^{rd} doses, respectively. By comparison, a significantly higher viability of R4GFP cells was observed for F4 transfection compared to lipofectamine after the 2^{nd} and 3^{rd} doses (p<0.001), but not after the 1^{st} dose. With regard to toxicity, IFN did not show any toxic effects, whereas the F5 formulation entrapping control siRNA showed slight toxicity to the cells (i.e. viability was ≧90% at different dose points). This was assumed to be due to the presence of lipid components, rather than siRNA itself in F5.

Characterization of sonicated siRNA nanosomes (B1-B9)—particle size, morphology, and zeta potential: The particle size data shown in Table 4 indicate that when the siRNA nanosomes were sonicated for a brief time period (30 seconds; B2), their size (122±26 nm; 70% confidence) was significantly reduced compared to the non-sonicated formulation (B1) (211±19; 70% confidence). The particle size was observed to gradually decrease with increasing sonication time periods. The smallest particles were observed in B5 (3 minute sonication), B6 (4 minute sonication) and B7 (5 minute sonication) which had average sizes of 103±14, 101±18 and 99±17 nm (70% confidence). The particles were found to be slightly larger when the sonication time period exceeded 5 minutes (i.e. B8 and B9 formulations). The zeta potential of all these formulations was also measured (Table 4). The data indicates that the zeta potential of different sonicated formulations did not change significantly compared to their respective non-sonicated counterparts. The average zeta potential of the sonicated formulations ranged from +11 to +14 mV and was comparable to that of the non-sonicated formulation (B1; ˜13.6±3.8 mV).

TABLE 4
Particle size and zeta potential of different
sonicated siRNA-nanosomes (B1-B9).

| | Sonication | | | | Zeta |
|---|---|---|---|---|---|
| | time | Particle size (nm)*/ | | | potential |
| Nanosome | (min) | 30% | 70% | 90% | (mV) |
| B1 | 0.0 | 76 ± 13 | 211 ± 19 | 405 ± 67 | 13.6 ± 3.8 |
| B2 | 0.5 | 47 ± 7 | 122 ± 26 | 269 ± 87 | 13.1 ± 1.8 |
| B3 | 1.0 | 65 ± 24 | 140 ± 44 | 275 ± 96 | 14.4 ± 3.2 |
| B4 | 2.0 | 50 ± 11 | 108 ± 28 | 240 ± 84 | 14.1 ± 3.2 |
| B5 | 3.0 | 43 ± 5 | 103 ± 14 | 196 ± 31 | 13.7 ± 3.8 |
| B6 | 4.0 | 42 ± 3 | 101 ± 18 | 192 ± 43 | 13.6 ± 3.1 |
| B7 | 5.0 | 42 ± 5 | 99 ± 17 | 186 ± 37 | 13.1 ± 3.2 |
| B8 | 6.0 | 52 ± 17 | 129 ± 41 | 237 ± 75 | 13.1 ± 3.5 |
| B9 | 7.0 | 43 ± 5 | 112 ± 19 | 225 ± 51 | 11.1 ± 3.7 |

*/Each column under “Particle size (nm)” states the percentage of nanosomes at or below indicated size. For example, for the unsonicated nanosomes, “B1,” 30% of the population was 76 nm or smaller, ±12 nm, 70% of the population was 211 nm or smaller, ±19 nm, and 90% of the population was 405 nm or smaller, ±67 nm.

siRNA encapsulation and integrity in the various sonicated formulations: Since the nanosomes (B1-B9) were exposed to different degrees of sonication, it was considered important to determine whether sonication had any deleterious effects on the integrity and encapsulation efficiency of siRNA within the nanosomes. This was assessed by measuring the siRNA level in both the encapsulated and free states using the RiboGreen® RNA quantitation assay and agarose gel electrophoresis. The non-sonicated formulation B1 was observed to entrap high levels of siRNA (76±3%), while leaving a small quantity of free unbound siRNA (6.3±0.5%) in the solution. When the formulations were sonicated, some of the formulations seemed to lose some of the siRNA, though none of them differed significantly (p>0.05) from the non-sonicated formulation (B1). The siRNA encapsulation efficiency was found to be 77±2% for B2, 76±6% for B3, 73±4% for B4, 72±3% for B5, 73±2% for B6, 72±4% for B7, 70±4% for B8 and 68±3% for B9. In contrast, the level of free siRNA in the sonicated formulations increased slightly compared to the non-sonicated formulation (B1). The free siRNA level was determined to be 7.8±0.2% for B2, 9.0±1.4% for B3, 9.8±0.5% for B4, 9.4±1.0% for B5, 9.2±1.2% for B6, 10.3±1.8% for B7, 9.9±0.7% for B8 and 10.2±0.8% for B9. The presence of protamine sulphate and trehalose in the nanosomes, and the decomplexing agent (i.e. SDS) likely influenced the fluorescence reading that resulted in the reduction of total siRNA amounts measured from both encapsulated and free state in each formulation. This study clearly indicates that sonication has no significant detrimental effect on the encapsulation efficiency of siRNA within the nanosomes.

The integrity of the encapsulated siRNA during this prolonged sonication time period was also assessed by subjecting the siRNA isolated from different samples (B1-B9) to agarose gel electrophoresis. The results shown in FIG. 5B indicate that all the siRNAs were released from the particles by 1% SDS treatment and there were no smearing of bands or any broken bands observed in any of those formulations. This study also confirms that extended sonication has no deleterious effects on the integrity of encapsulated siRNA in the nanosomes.

Tissue distribution of siRNA nanosomes following IP delivery: In order to assess the impact of particle size on the in vivo delivery, B1 (non-sonicated) and B6 (sonicated) were delivered via the IP route. Blank nanosomes containing no siRNA were also injected for comparison. The results indicate that most of the particles in the B6 formulation were deposited in the liver. A small percentage was also found in the spleen. However, there was nearly no deposition observed in the kidney, lung or heart. In contrast, when B1 was administered via the IP route, a significant reduction of particle deposition was observed in the liver. There was some deposition in the spleen, but none in the heart, kidney or lung.

In vitro and in vivo functional inhibition of GAPDH by siRNA nanosomes:

In vitro and in vivo GAPDH inhibition studies were performed to examine the ability of the siRNA nanosomes to effectively silence GAPDH, and also to compare functional inhibition by sonicated versus non-sonicated formulations. The results from the in vitro study indicate that both the B1 and the B6 formulations inhibited GAPDH at 100 nM siRNA. Furthermore, at the higher level of siRNA (i.e. 150 nM), a near complete inhibition of GAPDH was observed. It was also observed that the inhibition of GAPDH by the sonicated formulation (B6) was comparatively higher than that by the non-sonicated formulation (B 1). Similarly, when this experiment was conducted in vivo, the reduction of GAPDH was observed to be significantly higher with the sonicated formulation (B6) compared to the non-sonicated formulation (B1). These findings were consistent for both routes of delivery, i.e. the IP as well as the IV routes. The actual percentage inhibition of GAPDH by B6 via the IP and IV routes was 45±10% and 65±4%, respectively, whereas the inhibition by B1 via the IP and IV routes was 24±11% and 33±6%, respectively. In this study, the inhibition of GAPDH by B6 was significantly higher by IV delivery compared to the IP delivery. The results of the H&E staining of frozen sections of liver tissue and immunostaining of GAPDH in the same tissue sections indicate that the knock-down of GAPDH occurred mostly in the liver hepatocytes. Similar to the WB results, the knock-down efficiency of GAPDH in the liver hepatocytes was also observed to be higher for B6 compared to B1 when assessed by immunocytochemical analysis.

### Example 9

This Example discusses the results reported in the preceding Example.

RNAi offers great clinical potential as a therapeutic modality for a variety of viral diseases, especially HCV. For inhibition of HCV by RNAi to be effective, it would be useful to have a gene delivery vehicle which can deliver highly specific siRNA sequences to the virally infected liver hepatocytes. This study focused on the development of siRNA delivery vehicles which can specifically inhibit HCV replication and were tested in an in vitro cell culture model. As a proof of principle, the siRNA nanosomes were assessed for their delivery and knock-down of GAPDH specifically in liver hepatocytes in an in vivo mouse model.

In the in vitro study, the cell viability was unaltered or barely affected when the cells are transfected with siRNA alone or with siRNA encapsulated nanosomes at lower lipid-to-siRNA ratios (i.e. ≦5.34:1) for single or multiple treatment. When the lipid amount was increased, the viability of the cells was reduced. However, at lipid-to-siRNA ratios ≦5.34:1, the inhibition of HCV was significantly lower than that observed with a lipid-to-siRNA ratio ≧10.56:1. These findings led to the conclusion that a relatively higher lipid-to-siRNA ratio 13.36:1 (F4) provided a balance of viral inhibition (87%) with limited cytotoxicity (˜11%) after treatment with the first dose. We further attempted to determine whether long term treatment with siRNA formulations could eradicate HCV, given that a single treatment could not clear all the HCV from the cells. After three consecutive treatments with the formulations tested in the previous Example, complete clearance of HCV from the cells was not achieved. This observation suggested that delivering more than one siRNA at a time would be helpful in inducing long term inhibition of HCV replication. Cells treated with the formulations discussed herein had significantly higher viability than did cells contacted with lipofectamine; the formulations therefore provide the option for delivery of siRNA over longer periods if necessary in the treatment of HCV.

Several articles published recently have suggested that the particle size of a delivery system should not exceed 150 nm for successful targeting to the liver hepatic cells. (Fumoto et al., Pharm. Res. 20:1452-1459 (2003); Higuchi et al., Bio. Pharm. Bull. 29:1521-1523 (2006)). This is because the endothelial fenestrae have an approximate diameter of 150-175 nm (Braet and Wisse, Comp. Hepatol. 1:1 (2002)). In the non-sonicated formulation B 1, approximately 52% of particles are ≧150 nm If this formulation is selected for in vivo delivery, this amount of particles (˜52%) would not theoretically be able to enter into the liver hepatocytes, representing a significant clinical drawback. In the current study, rather than changing the composition, the formulation (F4) was sonicated to create smaller particles. Our study demonstrates that with as little as 30 seconds of sonication, the particles in B2 became significantly smaller (70% ˜122±26 nm) than the non-sonicated formulation B1 (70% ˜211±19 nm). We observed that only 30% of particles have a similar size distribution across the different batches (Table 3). Differences in particle size became clearly apparent when majority of the particles were counted (i.e. 90%). The results demonstrate that a relatively lower percentage of small particles are produced spontaneously during HPH and siRNA nanosome preparation. After the initial preparation step, the siRNA nanosomes may collide with each other with differing degrees of force and fuse together to form larger particles. The subsequent sonication of the formulation helps to separate these fused particles leading to a homogeneous distribution of smaller particles. Our study shows that a brief sonication of the formulation for a period of 2 to 5 minutes results in the formation of a homogeneous siRNA nanosome with smaller particles. However, it was also observed that sonication in excess of 5 minutes leads to an increase the particle size. The localized elevated temperature generated by prolonged sonication may be responsible for disrupting the thermodynamic environment of the saturated particles (Maulucci et al., Biophys J 88L3545-3550 (2005)), leading to the formation of larger particles. In a separate note, our study demonstrates that neither the siRNA's encapsulation efficiency nor the encapsulated siRNA's integrity was significantly affected by prolonged sonication, due perhaps to protection by protamine sulfate. (Kundu et al., J. Pharm. Pharmacol. 62:1103-1111 (2011); Dunne et al., J. Control. Release 92:209-219 (2003); Li et al., Gene Ther 5:930-937 (1998)).

The functional aspects of siRNA nanosomes require not only their successful entry into the cell cytoplasm, but also their escape from the endosomal compartments and their ability to subsequently reach the RISC factors. Our study shows that the siRNA nanosomes are initially engulfed by the endosomes and then carried into the cell cytoplasm by endosomes within 2 hours of transfection. Though we have not examined the release of siRNA nanosomes from the endosomes and the subsequent break down of the particles to free siRNA, this process could be explained by the ion pair model proposed by Xu and Szoka (Biochem 35:5616-5623 (1996). The model states that the interaction of cationic lipids with anionic lipids in the endosomes may be responsible for the quick entry and release of siRNA nanosomes from the endosomes. Also, as suggested by Tseng et al. (Drug Deliv Rev. 61:721-731 (2009), the near complete release of siRNA from the siRNA nanosomes into the cytoplasm within 12 hours of transfection is largely due to the presence of the cationic peptide (i.e. PS) within the nanosomes which helped in de-assembling siRNA from the complex and increasing the ion pair effects for quick endosomal escape. The release of siRNA from the siRNA nanosomes was also confirmed in this study by the functional gene silencing activities of siRNA nanosomes in inhibiting HCV replication in vitro and GAPDH reduction both in vitro and in vivo.

Delivery of siRNA encapsulated nanoparticles in vivo largely depends on the delivery technique. In this context, Larson et al. (Surgery, 142:262-269 (2007)) conducted a comparative study of siRNA delivery by different routes, and concluded that the deposition of siRNA encapsulated nanoparticles within different organs (liver, kidney, lung, spleen, bone and heart) differ based on the delivery route. Their results indicate that there is high siRNA uptake by the spleen and weak uptake by the liver when siRNA nanoparticles are administrated via the IP route. Our results contradict these findings. We observed high siRNA deposition in the liver and weak uptake by the spleen following IP delivery). In this context, as noted above, Fumoto 2003 and Higuchi 2006 reported that if the particles are larger than 141 nm, they will be unable to enter into the liver hepatocytes. Since our non-sonicated siRNA nanosomal particles (B1) are comparatively larger (70% ˜211±19); they were only minimally taken up by the liver hepatocytes. In contrast, as the majority of the particles in our sonicated siRNA nanosomes (B6) became smaller (70% ˜101±18 nm), they were deposited in significant amounts in the liver hepatocytes. Since the Larson et al. report did not indicate the optimal particle size of their siRNA nanoparticles, we hypothesize that the weak deposition of siRNA in the liver by IP delivery in that study was due to the larger particle size of siRNA complexes (>150 nm). Our findings indicate that if the particle size is maintained <150 nm, they will be deposited in the liver hepatocytes following either IP and IV delivery. These findings were also confirmed by the significant reduction of GAPDH in the liver hepatocytes by the sonicated nanosomes (B6), compared to their non-sonicated counterpart (B1).

Immunogenicity or non-specific side effects of gene delivery vehicles are major issues confronting clinical applications. In general, the type or composition of lipids, overall surface charge and the ratio of lipid to siRNA used in the formulation influences the overall toxicity. siRNA containing DOTAP lipoplex (Ma et al., Biochem. Biophys. Res. Comm 330:755-759 (2005) as well as siRNA containing PEI or PLL polycations [42] have been reported to induce IFN response and cytokine release. Recently, Semple et al. (Nature Biotechnol. 28:172-176 (2010)) reported that rationally designed ionizable cationic lipids of stable nucleic acid lipid particles (SNALP) can maintain significant siRNA knock-down efficiency, while rendering well tolerance of high doses by small animals (i.e. rats) and non-human primates (i.e. monkeys) with no obvious treatment-related changes in animal appearance or behavior. Similarly, in our in vivo delivery of siRNA nanosomes to mice, no abnormal physical behavior, or depression of the mice was noticed, nor were there any abnormal clinical changes in the liver tissue as determined by histochemical analysis (H&E).

The delivery of nanoparticles to the liver hepatocytes can be further enhanced by conjugating the particles with liver-specific antibodies which can specifically recognize and bind to the ligands on the hepatocyte cell surface, such as asyloglycoprotein receptors (ASGP-R). Fumoto et al. (Pharm. Res. 20:1452-1459 (2003)) have reported that the delivery of ASGP-R recognizable nanoparticles to the liver hepatic cells will be diminished if the particles are larger than 150 nm. The methods taught herein show how to reduce average particle size below 150 nm. Conjugating these particles with ASGP-R recognizing antibodies should further enhance the overall delivery of the siRNA encapsulated nanosomes to liver hepatocytes.

### Example 10

This Example sets forth a clinical trial regarding the administration of siRNA-nanosomes to subjects infected with HCV.

Participants in this study must have HCV. The HCV genotype of each participant will be determined. Participation in the initial trial will be limited to persons with HCV genotypes 1a, 1b, and 2a to reduce variability.

Before treatment begins, participants will have a physical exam. Blood (about 2 tablespoons) and urine tests will be performed. Women able to have children will have a blood pregnancy test. If a pregnancy test comes back positive, and for any reason the subject and/or the research staff believes that this may be an error, additional tests may be done to confirm or rule out pregnancy. Levels of HCV in the blood will be determined by conventional techniques.

A treatment cycle on this study is 4 weeks. Participants will receive pre-medications of dexamethasone and diphenhydramine prior to the infusion of siRNA-DOTAP:Chol nanosomes to lessen potential reactions to the infusion. The participant will receive a short infusion of siRNA-DOTAP:Chol by vein once every 7 days for 4 weeks. Participants will be examined by their doctor before each treatment. In addition, participants will return to the clinic on days 2, 3, and 4 after the first dose to have blood tests done, their vital signs checked, and to look for side effects. After every two treatment cycles, the participant's HCV levels will be measured. Participants can continue to receive treatments until side effects become too severe, or a maximum of 4 treatments have been given. If HCV levels have fallen below detectable levels at the end of 4 treatments, participants will return weekly for 6 months to determine the duration of the response. If HCV levels have not fallen below detectable levels at the end of 4 weeks, but have dropped by at least 50%, treatment may continue for another cycle of 4 weeks to see if levels can be reduced below detectable levels. Participants will return to the clinic 3 weeks after their last dose of siRNA-DOTAP:Chol to have their vital signs checked and to look for side effects, in addition to having viral levels checked.

Participants entered at a given dose level will not be able to receive a higher dose while on study. A group of 3 participants will receive siRNA-DOTAP:Chol by vein at each dose level. After treating 3 participants at a given dose level, the participants will be observed for 2 weeks to evaluate the toxicity. The information showing if the participants develop severe side effects, referred to as dose-limiting toxicity (DLT), will be recorded for computing the chance of toxicity. This information will be used to help select the dose level for the next group of participants. The goal is to find the dose level where 10% of participants develop severe side effects (dose-limiting toxicity). Dosing will start at 0.01 milligrams per kilogram. It is anticipated the maximum dose tested will be 0.09 milligrams per kilogram.

All the participants will be treated in a dose-escalation fashion starting from the lowest level. The next dose level can be moved up if calculation of the side effects shows that a higher dose is needed. However, no skipping of doses will be allowed.

Inclusion Criteria:

HCV infection verified to be of HCV genotype 1a, 1b, or 2a.

Negative serum pregnancy test (serum HCG) if female and of childbearing potential. Subjects must agree to practice effective birth control during the study period.

Negative serology for Human Immunodeficiency Virus.

Patients must be >1=4 weeks beyond major surgical procedures, and must be >/=1.5 weeks beyond minor surgical procedures, and must not have evidence of wound dehiscence, active wound infection, or comparable major residual complications of the surgery. Prothrombin time (PT) and Partial thromboplastin time (PTT) <1.25 times the institutional upper limit of normal.

Adequate renal function documented by serum creatinine of <=1.5 mg/dl or calculated creatinine clearance >50 ml/min

Adequate hepatic function as documented by serum bilirubin<1.5 mg/dl and SGOT and SGPT </=1.5 * upper limit of normal.

Stable cardiac condition with a left ventricular ejection fraction >50%.

Patients must have voluntarily signed an informed consent in accordance with institutional policies.

Exclusion Criteria:

Females who are pregnant or breast-feeding.

Active systemic viral, bacterial or fungal infections requiring treatment.

Patients with serious concurrent illness or psychological, familial, sociological, geographical, or other concomitant conditions that, in the opinion of the investigator, would not permit adequate follow-up and compliance with the study protocol.

Use of any investigational agent within four weeks of study treatment.

Prior nucleic acid therapy.

History of myocardial infarction within 6 months, angina within the past 6 months, or a history of arrhythmias on active therapy.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A composition comprising at least a first and a second isolated small interfering RNA (siRNA), each siRNA comprising a sense sequence having a 5′ end and a 3′ end, and a complementary antisense sequence having a 5′ and a 3′ end, wherein said sense sequence of said first isolated siRNA comprises 19-24 contiguous nucleotides of a sequence selected from the group consisting of:
(a)
(SEQ ID NO: 29)
5′-GCCUUGUGGUACUGCCUGAUAGGG-3′;
(b)
(SEQ ID NO: 30)
5′-CCCCGGGAGGUCUCGUAGACCGUG-3′;
(c)
(SEQ ID NO: 31)
5′-GAGGUCUCGUAGACCGUGCACCAU-3′;
(d)
(SEQ ID NO: 32)
5′-ACCGUGCACCAUGAGCACAAAUCC-3′;
(e)
(SEQ ID NO: 33)
5′-AACCUCAAAGAAAAACCAAAAGAA-3′;
and,
wherein said sense strand of said second isolated siRNA is selected from the group consisting of sequences (a)-(e),
provided that said sense sequence of said first isolated siRNA and said sense sequence of said second isolated siRNA are not the same.

2. The composition of claim 1, further wherein said sense sequence and said antisense sequence of said first siRNA and of said second siRNA, respectively, have a two T overhang on said 3′ ends.

3. The composition of claim 1, further wherein said sense sequence, with overhang, of said first siRNA consists of a sequence selected from the group consisting of SEQ ID NO:11, SEQ ID NO:15, SEQ ID NO:17, SEQ ID NO:19, and SEQ ID NO:23, provided that if said sense sequence selected is SEQ ID NO:17, the sense sequence selected for said sense sequence of said second siRNA is not SEQ ID NO:19.

4. The composition of claim 3, further wherein said sense sequence, with overhang, of said second siRNA consists of a sequence selected from the group consisting of SEQ ID NO:11, SEQ ID NO:15, SEQ ID NO:17, SEQ ID NO:19, and SEQ ID NO:23.

5. The composition of claim 1, further wherein said sense and antisense sequences of said first siRNA have the sequences SEQ ID NO:17 and SEQ ID NO:18, respectively, and said sense and antisense sequences of said antisense sequences of said second siRNA have the sequences of SEQ ID NO:23 and SEQ ID NO:24, respectively.

6. The composition of claim 1, further wherein said first siRNA and said second siRNA are condensed with protamine sulfate.

7. The composition of claim 1, wherein said first siRNA and said second siRNA are complexed to or encapsulated in a nanosome.

8. The composition of claim 7, wherein said nanosome comprises a cationic lipid.

9. The composition of claim 8, further wherein said cationic lipid is selected from the group consisting of 3β-[N-(N′,N′-dimethylaminoethane)-carbamoyl] cholesterol hydrochloride (DC-cholesterol); 1,2-dioleoyl-3-trimethylammonium-propane (DOTAP); 1,2-dioleoyl-3-dimethylammonium-propane (DODAP);
dimethyldioctadecylammonium (DDAB); 1,2-dilauroyl-sn-glycero-3-ethylphosphocholine (Ethyl-PC), or 1,2-di-O-octadecenyl-3-trimethylammonium propane (DOTMA).

10. The composition of claim 8, further wherein said cationic lipid is DOTAP.

11. The composition of claim 7, wherein said nanosome further comprises a neutral lipid.

12. The composition of claim 11, wherein said neutral lipid is cholesterol.

13. The composition of claim 7, wherein said nanosome comprises a sugar.

14. The composition of claim 13, wherein said sugar is selected from the group consisting of trehalose, sucrose, lactose and mannitol.

15. The composition of claim 14, wherein the sugar is trehalose.

16. The composition of claim 1, further wherein the composition is a powder.

17. The composition of claim 1, further wherein the composition is a lyophilized powder.

18. The composition of claim 1, further wherein the composition is a spray dried powder.

19. A method of inhibiting replication of hepatitis C virus (HCV) in a subject in need thereof, said method comprising co-administering to said subject a first and a second isolated small interfering RNA (siRNA), each siRNA comprising a sense sequence having a 5′ end and a 3′ end, and a complementary antisense sequence having a 5′ and a 3′ end, wherein said sense sequence of said first isolated siRNA comprises 19-24 contiguous nucleotides of a sequence selected from the group consisting of:
(a)
(SEQ ID NO: 29)
5′-GCCUUGUGGUACUGCCUGAUAGGG-3′;
(b)
(SEQ ID NO: 30)
5′-CCCCGGGAGGUCUCGUAGACCGUG-3′;
(c)
(SEQ ID NO: 31)
5′-GAGGUCUCGUAGACCGUGCACCAU-3′;
(d)
(SEQ ID NO: 32)
5′-ACCGUGCACCAUGAGCACAAAUCC-3′;
(e)
(SEQ ID NO: 33)
5′-AACCUCAAAGAAAAACCAAAAGAA-3′;
and,
wherein said sense strand of said second isolated siRNA is selected from the group consisting of sequences (a)-(e),
provided that said sense sequence of said first isolated siRNA and said sense sequence of said second isolated siRNA are not the same,
thereby inhibiting the replication of HCV in said subject.

20. The method of claim 19, further wherein said sense sequence and said antisense sequence of said first siRNA and of said second siRNA, respectively, have a two T overhang on said 3′ ends.

21. The method of claim 19, further wherein said sense sequence, with overhang, of said first siRNA consists of a sequence selected from the group consisting of SEQ ID NO:11, SEQ ID NO:15, SEQ ID NO:17, SEQ ID NO:19, and SEQ ID NO:23, provided that if said sense sequence selected is SEQ ID NO:17, the sense sequence selected for said sense sequence of said second siRNA is not SEQ ID NO:19.

22. The method of claim 20, further wherein said sense sequence, with overhang, of said second siRNA consists of a sequence selected from the group consisting of SEQ ID NO:11, SEQ ID NO:15, SEQ ID NO:17, SEQ ID NO:19, and SEQ ID NO:23.

23. The method of claim 22, further wherein said sense and antisense sequences of said first siRNA have the sequences SEQ ID NO:17 and SEQ ID NO:18, respectively, and said sense and antisense sequences of said antisense sequences of said second siRNA have the sequences of SEQ ID NO:23 and SEQ ID NO:24, respectively.

24. The method of claim 18, further wherein said first siRNA and said second siRNA are complexed to or encapsulated in a nanosome, thereby forming an siRNA-nanosome.

25. The method of claim 24, wherein said nanosome comprises a cationic lipid.

26. The method of claim 25, further wherein said cationic lipid is selected from the group consisting of 313-1N-(N′,N′-dimethylaminoethane)-carbamoyllcholesterol hydrochloride (DC-cholesterol); 1,2-dioleoyl-3-trimethylammonium-propane (DOTAP); 1,2-dioleoyl-3-dimethylammonium-propane (DODAP);
dimethyldioctadecylammonium (DDAB); 1,2-dilauroyl-sn-glycero-3-ethylphosphocholine (Ethyl-PC), or 1,2-di-O-octadecenyl-3-trimethylammonium propane (DOTMA).

27. The method of claim 25, further wherein said cationic lipid is DOTAP.

28. The method of claim 24, further wherein said nanosome comprises a neutral lipid.

29. The method of claim 28, further wherein said neutral lipid is cholesterol.

30. The method of claim 19, further wherein said first and said second siRNAs are condensed with a cationic polymer.

31. The method of claim 30, further wherein said cationic polymer is protamine sulfate.

32. The method of claim 24, further wherein said nanosome comprises a sugar.

33. The method of claim 32, wherein said sugar is selected from the group consisting of trehalose, sucrose, lactose and mannitol.

34. The method of claim 33, wherein said sugar is trehalose.

35. The method of claim 24, further wherein the siRNA-nanosome is a rehydrated powder.

36. The method of claim 24, further wherein the siRNA-nanosome is a rehydrated lyophilized powder.

37. The method of claim 24, further wherein the siRNA-nanosome is a rehydrated spray dried powder.

38. The method of claim 24, wherein said siRNA-nanosomes are sonicated within approximately 1½ hours prior to administration to said subject.

39. The method of claim 24, wherein said siRNA-nanosomes are sonicated within one hour prior to administration to said subject.

40. The method of claim 24, wherein said siRNA-nanosomes are sonicated 5-45 minutes before administration to said subject.

41. The method of claim 38, wherein said sonication is for less than 5 minutes.

42. The method of claim 38, wherein said sonication is for less than 3 minutes.

43. The method of claim 19, wherein said HCV is selected from the genotypes of type 1a, 1b, and 2a.

44. The method of claim 43, wherein said HCV is of genotype is 2a.

45. A method of preparing a nucleic acid agent for administration to a liver of a subject in need thereof, said method comprising
(a) providing a plurality of said nucleic acid agent complexed to or encapsulated in nanosomes, said nanosomes having an average size, and
(b) sonicating said plurality nucleic acid agent complexed to or encapsulated in said nanosome to reduce the average size of said nanosomes,
thereby preparing said nucleic acid agent for administration.

46. The method of claim 45, wherein said nucleic acid agent is an siRNA.

47. The method of claim 45, wherein said nucleic acid agent is an expression plasmid.

48. The method of claim 45, wherein said sonication is for less than 5 minutes.

49. The method of claim 45, wherein said sonication is conducted within 1.5 hours of administration of the nanosomes to said subject.

50. The method of claim 45, wherein said sonication is conducted 5-45 before administration of the nanosomes to said subject.

51. The method of claim 45, wherein said average size of a majority of said nanosomes is 150 nm or less.


SEQUENCE LISTING (48 sequences)

SEQ ID NO: 1 - DNA, 21 bp, Artificial Sequence
  Description of Artificial Sequence Synthetic oligonucleotide
gcgccuagccauggcguuatt

SEQ ID NO: 2 - DNA, 21 bp, Artificial Sequence
  Description of Artificial Sequence Synthetic oligonucleotide
uaacgccauggcuaggcgctt

SEQ ID NO: 3 - DNA, 21 bp, Artificial Sequence
  Description of Artificial Sequence Synthetic oligonucleotide
uugccgggaagacuggguctt

SEQ ID NO: 4 - DNA, 21 bp, Artificial Sequence
  Description of Artificial Sequence Synthetic oligonucleotide
gacccagucuucccggcaatt

SEQ ID NO: 5 - DNA, 21 bp, Artificial Sequence
  Description of Artificial Sequence Synthetic oligonucleotide
cccacucuaugcccggccatt

SEQ ID NO: 6 - DNA, 21 bp, Artificial Sequence
  Description of Artificial Sequence Synthetic oligonucleotide
uggccgggcauagagugggtt

SEQ ID NO: 7 - DNA, 21 bp, Artificial Sequence
  Description of Artificial Sequence Synthetic oligonucleotide
gacugcuagccgaguagcgtt

SEQ ID NO: 8 - DNA, 21 bp, Artificial Sequence
  Description of Artificial Sequence Synthetic oligonucleotide
cgcuacucggcuagcaguctt

SEQ ID NO: 9 - DNA, 21 bp, Artificial Sequence
  Description of Artificial Sequence Synthetic oligonucleotide
cguuggguugcgaaaggcctt

SEQ ID NO: 10 - DNA, 21 bp, Artificial Sequence
  Description of Artificial Sequence Synthetic oligonucleotide
ggccuuucgcaacccaacgtt

SEQ ID NO: 11 - DNA, 21 bp, Artificial Sequence
  Description of Artificial Sequence Synthetic oligonucleotide
cuugugguacugccugauatt

SEQ ID NO: 12 - DNA, 21 bp, Artificial Sequence
  Description of Artificial Sequence Synthetic oligonucleotide
uaucaggcaguaccacaagtt

SEQ ID NO: 13 - DNA, 21 bp, Artificial Sequence
  Description of Artificial Sequence Synthetic oligonucleotide
gggcgcuugcgagugcccctt

SEQ ID NO: 14 - DNA, 21 bp, Artificial Sequence
  Description of Artificial Sequence Synthetic oligonucleotide
ggggcacucgcaagcgccctt

SEQ ID NO: 15 - DNA, 21 bp, Artificial Sequence
  Description of Artificial Sequence Synthetic oligonucleotide
ccgggaggucucguagacctt

SEQ ID NO: 16 - DNA, 21 bp, Artificial Sequence
  Description of Artificial Sequence Synthetic oligonucleotide
ggucuacgagaccucccggtt

SEQ ID NO: 17 - DNA, 21 bp, Artificial Sequence
  Description of Artificial Sequence Synthetic oligonucleotide
ggucucguagaccgugcactt

SEQ ID NO: 18 - DNA, 21 bp, Artificial Sequence
  Description of Artificial Sequence Synthetic oligonucleotide
gugcacggucuacgagacctt

SEQ ID NO: 19 - DNA, 21 bp, Artificial Sequence
  Description of Artificial Sequence Synthetic oligonucleotide
cgugcaccaugagcacaaatt

SEQ ID NO: 20 - DNA, 21 bp, Artificial Sequence
  Description of Artificial Sequence Synthetic oligonucleotide
uuugugcucauggugcacgtt

SEQ ID NO: 21 - DNA, 21 bp, Artificial Sequence
  Description of Artificial Sequence Synthetic oligonucleotide
auccuaaaccucaaagaaatt

SEQ ID NO: 22 - DNA, 21 bp, Artificial Sequence
  Description of Artificial Sequence Synthetic oligonucleotide
uuucuuugagguuuaggautt

SEQ ID NO: 23 - DNA, 21 bp, Artificial Sequence
  Description of Artificial Sequence Synthetic oligonucleotide
ccucaaagaaaaaccaaaatt

SEQ ID NO: 24 - DNA, 21 bp, Artificial Sequence
  Description of Artificial Sequence Synthetic oligonucleotide
uuuugguuuuucuuugaggtt

SEQ ID NO: 25 - DNA, 21 bp, Artificial Sequence
  Description of Artificial Sequence Synthetic oligonucleotide
aaaccaaaagaaacaccaatt

SEQ ID NO: 26 - DNA, 21 bp, Artificial Sequence
  Description of Artificial Sequence Synthetic oligonucleotide
uugguguuucuuuugguuutt

SEQ ID NO: 27 - DNA, 21 bp, Artificial Sequence
  Description of Artificial Sequence Synthetic oligonucleotide
acguccaaguucgauuggctt

SEQ ID NO: 28 - DNA, 21 bp, Artificial Sequence
  Description of Artificial Sequence Synthetic oligonucleotide
gccaaucgaacuuggacgutt

SEQ ID NO: 29 - RNA, 24 nt, Artificial Sequence
  Description of Artificial Sequence Synthetic oligonucleotide
gccuugugguacugccugauaggg

SEQ ID NO: 30 - RNA, 24 nt, Artificial Sequence
  Description of Artificial Sequence Synthetic oligonucleotide
ccccgggaggucucguagaccgug

SEQ ID NO: 31 - RNA, 24 nt, Artificial Sequence
  Description of Artificial Sequence Synthetic oligonucleotide
gaggucucguagaccgugcaccau

SEQ ID NO: 32 - RNA, 24 nt, Artificial Sequence
  Description of Artificial Sequence Synthetic oligonucleotide
accgugcaccaugagcacaaaucc

SEQ ID NO: 33 - RNA, 24 nt, Artificial Sequence
  Description of Artificial Sequence Synthetic oligonucleotide
aaccucaaagaaaaaccaaaagaa

SEQ ID NO: 34 - RNA, 380 nt, Hepatitis C virus
gccagcccccugaugggggcgacacuccaccaugaaucacuccccuggaggaacuacugucuucacgcagaaagcgucuagccauggcguuaguaugagugucgugcagccuccaggacccccccucccgggagagccauaguggucugcggaaccggugaguacaccggaauugccagacgaccggguccuuucuuggauaaacccgcucaaugccuggagauuugggcgugcccccgcaagacugcuagccgaguaguguugggucgcgaaaggccuugugguacugccugauagggugcuugcgagugccccgggagguucguagaccgugcaccaugagcacgaauccuaaaccucaaagaaaaaccaaacguaac

SEQ ID NO: 35 - DNA, 420 bp, Hepatitis C virus
acctgcccctaataggggcgacactccgccatgaatcactcccctgtgaggaactactgtcttcacgcagaaagggcctagccatggcgttagtatgagtgtggtacagcctccagggcggcccctccggggagaggcatagtggtctgcggaaggggtgagtacacgggaattgccgggaagactggctgctttcttggataaaggcagtctatgcccggccatttgggcgtgcccgcgcaagactgctagccgagtagggttgggttgcgaaaggccttctggtactgcctgatagggcgcttgcgagtggcccgggaggtctggtagaccgtgcagcatgaggacaaatcctaaacctcaaagaaaaaccaaaagaaacaccaaccgtcgccgaatgattgaacaagatggattgca

SEQ ID NO: 36 - DNA, 21 bp, Artificial Sequence
  Description of Artificial Sequence Synthetic primer
tcttcacgcagaaagcgtcta

SEQ ID NO: 37 - DNA, 20 bp, Artificial Sequence
  Description of Artificial Sequence Synthetic primer
cggttccgcagaccactatg

SEQ ID NO: 38 - DNA, 14 bp, Artificial Sequence
  Description of Artificial Sequence Synthetic probe
tgcagcctccagga

SEQ ID NO: 39 - DNA, 21 bp, Artificial Sequence
  Description of Artificial Sequence Synthetic oligonucleotide
aataggggcgacactccgcca

SEQ ID NO: 40 - DNA, 21 bp, Artificial Sequence
  Description of Artificial Sequence Synthetic oligonucleotide
atcagagcagccgattgtctg

SEQ ID NO: 41 - DNA, 18 bp, Artificial Sequence
  Description of Artificial Sequence Synthetic primer
ctgtgaggaactactgtc

SEQ ID NO: 42 - DNA, 21 bp, Artificial Sequence
  Description of Artificial Sequence Synthetic oligonucleotide
ccucaaagaaaaaccaaaatt

SEQ ID NO: 43 - DNA, 21 bp, Artificial Sequence
  Description of Artificial Sequence Synthetic oligonucleotide
gcgccuagccauggcguuatt

SEQ ID NO: 44 - DNA, 23 bp, Artificial Sequence
  Description of Artificial Sequence Synthetic oligonucleotide
gaggtctggtagaccgtgcagca

SEQ ID NO: 45 - DNA, 23 bp, Artificial Sequence
  Description of Artificial Sequence Synthetic oligonucleotide
gaggtctggtagaccgtgcggca

SEQ ID NO: 46 - DNA, 27 bp, Artificial Sequence
  Description of Artificial Sequence Synthetic oligonucleotide
tagacctcaaagaaaaaccaagggaaa

SEQ ID NO: 47 - DNA, 27 bp, Artificial Sequence
  Description of Artificial Sequence Synthetic oligonucleotide
tagacctcaaagaaaaaccgagggaaa

SEQ ID NO: 48 - DNA, 27 bp, Artificial Sequence
  Description of Artificial Sequence Synthetic oligonucleotide
taaacctcaaaaaaaaaccaaaggaaa